(19) United States
(12) Patent Application Publication
(10) Pub. No.: US 2026/0318900 A1
(43) Pub. Date: Oct. 15, 2026

(54) SINGLE CELL SUPER-RESOLUTION PROTEOMICS

(71) Applicant: Massachusetts Institute of Technology, Cambridge, MA (US)

(72) Inventors: Mark Harnett, Medford, MA (US); Dimitra Vardalaki, Boston, MA (US)

(21) Appl. No.: 19/106,534

(22) PCT Filed: Aug. 25, 2023
(86) PCT No.: PCT/US2023/031184

§ 371 Date: Feb. 25, 2025

Related U.S. Application Data
(60) Provisional application No. 63/400,787, filed on Aug. 25, 2022.

(51) Int. Cl. G01N 33/483; G01N 33/575; G01N 33/68

(52) U.S. Cl. CPC G01N 33/4833; G01N 33/57557; G01N 33/6896

(57) ABSTRACT

Disclosed are methods for full morphological labeling of individual neurons from any species or cell type for subsequent cell-delineated protein analysis. These methods, which combines patch-clamp electrophysiology with epitope-preserving magnified analysis of proteome (eMAP), additionally allow for correlation of physiological properties with subcellular protein expression.

### RELATED APPLICATIONS

This application claims the benefit of priority to U.S. Provisional Patent Application Ser. No. 63/400,787, filed Aug. 25, 2022.

### BACKGROUND

Provided herein is high-contrast labeling of single, non-genetically modified cells for super-resolution proteomics. Super-resolution microscopy promises to reveal the subcellular spatial distributions of proteins, a key unknown in cell biology across fields, but this requires that single cells are labeled with sufficient contrast to delineate them from neighboring cells. If single cells are not effectively delineated, then proteins of interest, which are promiscuously labeled throughout the tissue, cannot be assigned to a given cell or even to subcellular compartments (e.g. the axon, a synapse, etc.). In dense and complex tissues like the brain, expression of an exogenous labeling protein is required for single cell contrast. Such labeling proteins are expressed via transgenes in genetically modified animals or via viruses. However, there are many species and cell types that are not amenable to such labeling strategies, particularly humans. The present disclosure allows for full morphological labeling of individual brain cells from any species or cell type for subsequent tissue expansion and cell-delineated subcellular proteomic analysis. This development allows for super-resolution proteomic imaging in the human brain with single cell morphological contrast for the first time, opening many new avenues for molecular investigation into human health and disease.

### SUMMARY OF THE INVENTION

The present disclosure starts with fresh brain slices from the species of interest. This is a long-standing technique in neuroscience that has been optimized for use in adult mammalian brains. An artificial cerebrospinal fluid and a detailed microdissection and slicing procedure is necessary to prepare the tissue. The slices are kept alive for >8 hours. Single neurons are then identified under a microscope in the living brain slices and patch-clamp electrophysiology is used to acquire intracellular access through a glass pipette. The patch-clamp pipette is filled with a specific concentration of biocytin, a monocarboxylic acid amide, which diffuses throughout the complicated morphological processes of the neuron. Filling a neuron with biocytin takes ~10 minutes. Electrophysiology can be conducted during this time and subsequently correlated with the resultant morphological structure and proteomic data from that cell. The pipette is carefully removed, the cell's membrane re-seals, and then the entire slice is placed in fixative. The tissue is then processed through a modified version of a MAP protocol (U.S. Provisional Patent Application 62/330,018, filed Apr. 29, 2016, now US 2019/0145868; incorporated by reference). Conventional tissue-expansion technologies for super-resolution imaging, including MAP and E×M, lose pre-expansion labels like biocytin due to structural changes imposed by the gelation steps. The modified MAP protocol that has been developed allows not only recovery of the pre-expansion biocytin signal but also to further amplify it. First, the strong bond of biocytin-streptavidin is used to link a fluorescence protein to the biocytin. This reaction can be effectively performed before the gelation step. A special fixation step is used to withstand gelation. One of MAP's methodological advantages is that multiple rounds of protein staining can be conducted. However, this requires destaining steps, where signals from the previous round of staining are eliminated. The biocytin signal is preserved since the streptavidin-biocytin complex has been chemically fixed prior to the original gelation step. This preserves the single cell structural contrast label through multiple rounds of staining. Multi-round staining allows investigation of the distributions of many different proteins in the same cell and its subcellular structures.

### BRIEF DESCRIPTION OF THE DRAWINGS

FIG. 1a is a schematic of experimental tissue processing for full morphological labelling of individual neurons and subsequent cell-delineated super-resolution proteomic analysis.

FIG. 1b (left column) Two-photon z-stack of a mouse L5 PC filled via somatic patch pipette with Alexa-488 and biocytin. (middle column) Confocal z-stack of the same neuron at the stage of intermediate expansion. Strepatvidin Alexa-fluor 488 reveals biocytin. (right column) Overlay of the two z-stacks.

FIG. 1c shows after intermediate expansion, the originally 300 μm thick mouse brain slice is resliced in thinner slices. Confocal z-stacks of the 4 slices which contain the neuron shown in FIG. 1b.

FIG. 1d shows confocal z-stacks of 3×-expanded soma and branches highlighted in FIG. 1c.

FIG. 1e shows a magnified view of basal branch from d. Green arrowheads indicate example spines shown to the right of the branch. From left to right: image of the cell-filling biocytin channel stained with Strepatvidin Alexa-fluor 488, presynaptic protein Bassoon stained with anti-guinea pig-Alexa405 (blue), NMDAR subunit NR1 stained with anti-mouse-Alexa555 (yellow), and AMPAR subunit GluR1 stained with anti-rabbit-Alexa647 (red).

FIG. 2a shows a two-photon z-stack of a human L2/3 PC filled via somatic patch pipette with Alexa-488 and biocytin.

FIG. 2b shows confocal z-stack of the same neuron at the stage of intermediate expansion. Streptavidin Alexa fluor 488 reveals biocytin.

FIG. 2c shows re-slicing of the processed human brain slice resulted in thinner slices. Confocal z-stacks of the 4 slices that contain the neuron shown in FIG. 2b.

FIG. 2d shows confocal z-stacks of 3x expanded dendritic branch highlighted in FIGS. 2a, 2b, and 2c.

FIG. 2e shows an example basal branch from a different human neuron. Dendritic spine of interest indicated by green box.

FIG. 2f shows a magnified view of dendritic spine from FIG. 2e.

FIG. 2g shows multi-round staining images of a processed human tissue. Biocytin channels from both rounds were used as the common channel for image registration. (top) Round 1: Bassoon (blue), GluN1 (yellow), and GluA1 & GluA2 (red). (bottom) Round 2: Shank3 (green), α-synuclein (magenta), and PSD-95 (cyan).

FIG. 3a shows (top right) Two-photon image of a human spine filled via somatic patch pipette with Alexa-488 and biocytin (top middle) individual (grey) and average (colored) voltage traces recorded in current clamp mode at the soma in response to glutamate uncaging at the spine in control aCSF (top left) same as (top middle) after washing in of Mg²⁺-free plus 20 μM DNQX aCSF. (bottom) confocal image of the same spine after 3× expansion. From left to right: image of the cell-filling biocytin channel stained with Streptavidin Alexa-fluor 488, AMPAR subunit GluR1 and GluR2 stained with anti-rabbit-Alexa647 (red) and NMDAR subunit NR1 stained with anti-mouse-Alexa555 (yellow).

FIG. 3b shows a confocal z-stack of a human PC at the stage of intermediate expansion. Branch of interest is indicated by green box.

FIG. 3c shows (left) 2-photon image of the branch from FIG. 3b. Numbers indicate the uncaging locations at individual spines. (right) example averaged voltage traces for spines 3, 5 & 7 in response to glutamate uncaging in control aCSF (red) and after washing in of Mg²⁺-free plus 20 μM DNQX aCSF (yellow).

FIG. 3d shows (left) confocal image of the same branch after ~3× expansion through Patch2MAP processing. (right) AMPAR subunit GluR1 and GluR2 stained with anti-rabbit-Alexa647 (red) and NMDAR subunit NR1 stained with anti-mouse-Alexa555 (yellow) superimposed to cell-filling biocytin channel stained with Streptavidin Alexa-fluor 488 for spines 3, 5 & 7.

FIG. 3e shows dots, correlation of mean AMPAR/NMDAR EPSP ratio and AMPAR/NMDAR signal intensity ratio; numbers correspond to the spines shown in FIGS. 3c and 3d. The data are fit with a line of slope 0.4735. (r) correlation coefficients, (P) P value.

FIG. 3f shows a confocal image of a human PC at the stage of intermediate expansion. Branch of interest is indicated by green box.

FIG. 3g shows (top) 2-photon image of the branch from FIG. 3f, (bottom) Confocal image of the same branch after ~3× expansion.

FIG. 3h (left) example averaged voltage traces for spines 1, 2 & 3 from 3g in response to glutamate uncaging in control aCSF (red) and after washing in of Mg²⁺-free plus 20 μM DNQX aCSF (yellow). (right) AMPAR subunit GluR1 and GluR2 stained with anti-rabbit-Alexa647 (red) and NMDAR subunit NR1 stained with anti-mouse-Alexa555 (yellow) superimposed to cell-filling biocytin channel stained with Streptavidin Alexa-fluor 488 for for spines 1, 2 & 3 from FIG. 3g.

FIG. 3i shows dots, correlation of mean AMPAR/NMDAR EPSP ratio and AMPAR/NMDAR signal intensity ratio; numbers correspond to the spines shown in FIG. 3g. The data are fit with a line of slope 0.16576. (r) correlation coefficients, (P) P value.

FIG. 3j shows dots, correlation of mean AMPAR/NMDAR EPSP ratio and normalized AMPAR/NMDAR signal intensity ratio (n=76 spines, 11 cells, 4 humans). The data are fit with a line of slope 0.33. (r) correlation coefficients, (P) P value.

FIG. 4a shows expansion ratios for human cells at the intermediate expansion step and at the final expansion step (n=13 cells, 5 humans).

FIG. 5a shows a schematic of analysis pipeline for calculating the AMPAR/NMDAR intensity for each synapse and the corresponding control.

FIG. 5b shows correlation values of electrophysiological to antibody intensity ratio from data are above the 100^{th} percentile (z-score=5.6622) of 1000 control.

FIG. 5c shows correlation values of electrophysiological to antibody intensity ratio from data are above the 100^{th} percentile for 10000 randomly drawn pairs from the data (z-score=5.6403).

FIG. 6a shows an example of a spine with signs of photodamage. (left) 2-photon images of the spine (orange arrowhead) before and after the initiation of uncaging experiment. (right) confocal image of the same spine in the expanded tissue. Notice the spinules arising from the spine (red arrowheads).

FIG. 6b shows an example of a spine where super-resolution revealed that two spines where targeted by glutamate uncaging instead of one. (left) 2-photon image of the targeted spines (orange arrowhead) (right) confocal images of the same spines in the expanded tissue showing the distinct necks (red lines) and heads (red arrowheads) of the two spines

FIG. 6c shows an example of uncaging location that did not correspond in a distinct spine in the expanded branch. (left) 2-photon image of the target location (orange arrowhead). (right) confocal image of the same branch in the expanded tissue. No corresponding spine is found (red arrowhead).

FIG. 6d shows the distribution of all the AMPAR/NMDAR for uEPSPs (left) and antibody signal intensity (right). Exclusions highlighted in color. Box plot represents median and IQR with whiskers extending to the most extreme points not considered outliers.

FIG. 6e shows dots, correlation of mean AMPAR/NMDAR EPSP ratio and normalized AMPAR/NMDAR signal intensity ratio.

FIG. 7a shows dots, correlation of mean AMPAR/NMDAR EPSP ratio and AMPAR/NMDAR signal intensity ratio. (r) correlation coefficients, (P) P value.

FIG. 8 shows the patient information. Male (M), female (F), right (R), left (L), temporal lobe (TL), frontal lobe (FL), anterior temporal lobe (ATL), brivaracetam (BRV), lamotrigine (LTG), lacosamide (LCM), lorazepam (LOR), oxcarbazepine (OCB), glioblastoma multiforme (GBM), isocitrate dehydrogenase (IDH) wildtype (wt), O6-826 methylguanine-DNA methyltransferase (MGMT), epidermal growth factor receptor (EGFR), cyclin 827 dependent kinase inhibitor 2A (CDKN2A).

FIG. 9a shows MRI-FLAIR image of a patient with GBM (white arrow) who received 5-aminolevulic acid (5-ALA) preoperatively.

FIG. 9b shows a two-photon single-plane image of acute brain slice from the border of the tumor from the patient in FIG. 9a containing labeled cancer cells (cell of interest indicated with arrow). The nucleus is not filled by 5-ALA.

FIG. 9c shows a two-photon z-stack and example spontaneous somatic voltages of labeled cancer cell from FIG. 9b filled via somatic patch pipette with Alexa-488 and biocytin. Spontaneous oscillations (upper trace) and magnified faster membrane depolarizations (bottom trace, inset).

FIG. 9d shows confocal z-stacks of re-sliced and expanded cancer cell from FIGS. 9b and 9c.

FIG. 9e shows synaptic nanoclusters formed on the membrane of the cancer cell that are consistent with functional synapses. Nanoclusters contain NMDA receptors (GluN1), AMPA receptors (GluA1 & GluA2) and Bassoon. Scale bars correspond to the physical dimensions of the tissue at the time of the imaging without accounting for the level of tissue expansion.

FIG. 10a shows a schematic of experimental tissue processing for expansion-based super-resolution imaging.

FIG. 10b shows an example confocal image of a Thy1-GFP+L5 pyramidal neuron after 4× expansion of an originally 45 μm thick slice. Scale bar: 200 μm expanded/ 50 μm original.

FIG. 10c shows an example confocal image of a L5 pyramidal neuron dendritic segment after 4× expansion. Arrowheads indicate filopodia. Arrows indicate example spine and filopodium shown in FIG. 10e. Scale bar: 5 μm expanded/1.25 μm original.

FIG. 10d is an illustration of criteria to classify of dendritic protrusions as spines or filopodia.

FIG. 10e is a magnified image of a spine and a filopodium from FIG. 10c (arrows). Scale bar 2 μm expanded/0.5 μm original.

FIG. 10f is a histogram of dhead/dneck (head/neck diameter ratios) across all dendritic protrusions (n=2234). Shaded area indicates dhead/dneck <1.3, one of the criteria used to classify protrusions as filopodia.

FIG. 10g is a fraction of dendritic protrusions classified as filopodia (L5: n=2234 dendritic protrusions from 123 dendritic branches from 4 mice; L2/3: n=442 dendritic protrusions from 13 dendritic branches from 3 mice). Box plot represents median and IQR with whiskers extending to the most extreme points not considered outliers. P=0.33, Kruskal-Wallis test.

FIG. 11a shows an illustration of a dendritic protrusion and corresponding measurements: head diameter (dhead), neck diameter (dneck), length (l).

FIGS. 11b-11d shows population histograms of morphological characteristics across all dendritic protrusions (n=2234).

FIG. 11e shows a population histogram of the relationship between dhead and dneck. Shaded area indicates a ratio below 1.3, the first criterion used to classify filopodia versus spines.

FIG. 11f shows a population histogram of dhead/l for protrusions with dhead/dneck below 1.3 (shaded area in FIG. 11e). Protrusions with dhead/l above 3 were classified as filopodia, those below 3 were likely short stubby spines and were not analyzed further (shaded area=(dhead/dneck<1.3)∩(l/dhead>3)).

FIG. 11g shows the population histogram for each of the four mice.

FIG. 11h shows the fraction of dendritic protrusions classified as filopodia per mouse. Box plot represents median and IQR with whiskers extending to the most extreme points not considered outliers. ns P=0.093, Kruskal-Wallis test.

FIG. 12a shows an example confocal image of a V1 L2/3 neuron (green arrowhead) expressing GFP after viral transfection in V1 in an originally 45 μm thick slice. Scale bar: 100 μm expanded/59 μm original. Image was taken after reshrinking the tissue from 4× expansion to 1.7× expansion.

FIG. 12b shows a Box plot (left) and distribution (right) of signal intensity in Bassoon, NMDAR and AMPAR channels for L2/3 pyramidal neuron spines (n=275). Box plot represents median and IQR with whiskers extending to the most extreme points not considered outliers. Signal in each channel is shown for all dendritic protrusions, each represented by one dot.

FIG. 12c shows a similar box plot as 12b, but for filopodia (n=134).

FIG. 13a shows cumulative density function of Bassoon signal intensity in spines and filopodia. Vertical line at the choosen threshold (anti-Bassoon signal=0).

FIG. 13b shows a magnified plot of a around 0.

FIGS. 13c and 13d show an example anti-Bassoon signal intensities of ~4 a.u. for a filopodium below 13c and another filopodium above 13d the threshold.

FIG. 14a shows an example four channel images of a spine (top) versus a filopodium (bottom), from FIG. 10. From left to right: image of the cell-filling GFP channel stained with anti-chicken-Alexa488, presynaptic protein Bassoon stained with anti-guinea pig-Alexa405, NMDAR subunit NR1 stained with anti-mouse-Alexa555, and AMPAR subunit GluR1 stained with anti-rabbit-Alexa 647. Scale bar 2 μm expanded/0.5 μm original.

FIG. 14b shows a box plot (left) and distribution (right) of signal intensity in Bassoon, NMDAR, and AMPAR channels for spines (n=1505). Box plot represents median and IQR with whiskers extending to the most extreme points not considered outliers. Signal in each channel is shown for all dendritic protrusions, each represented by one dot. nsP=0.1331, ****P<0.0001, Kruskal-Wallis test.

FIG. 14c shows a similar box plot and distribution, but for filopodia (n=614). ****P<0.0001, Kruskal-Wallis test.

FIG. 15 shows example four channel images of dendritic protrusions with different dhead/dneck values (increasing from left to right). From top to bottom: cell-filling GFP stained with anti-Chicken Alexa488 at lower magnification to show full protrusion shape, presynaptic protein Bassoon stained with anti-Guinea pig-Alexa405, NMDAR subunit NR1(GluN1) stained with anti-Mouse-Alexa555, and AMPAR subunit GluR1(GluA1) stained with anti-Rabbit-Alexa647, all at higher magnification to show synaptic localization. Scale bars indicate native tissue scale of 0.5 μm, which corresponds to 2 μm final expansion imaged scaled.

FIG. 16a shows anti-GluA1 signal intensity as a function of head diameter for spines and filopodia.

FIG. 16b shows the correlation between head diameter and anti-GluA1 signal intensity for spines. The data are fitted with a line of slope 421+/−22 using linear regression.

FIG. 16c shows the correlation between diameter of filopodium head and anti-GluaA1 signal intensity. The data are fitted with a line of slope 95+/−39 using linear regression. Correlation coefficients (r) and p-values were obtained from a two-tailed, non-parametric Spearman correlation.

FIG. 17a shows an example two-photon image of a basal dendritic branch from a L5 V1 pyramidal neuron filled with Alexa-488, showing spines and a filopodium. Two-photon glutamate uncaging locations are indication at a test spine and a filopodium.

FIG. 17b shows voltage traces recorded at the soma in response to two-photon glutamate uncaging at a representative spine (top) and filopodium (middle), as well as the same filopodium in Mg²⁺-free ASCF with AMPARs blocked (DNQX, 20 μM; bottom).

FIG. 17c shows voltage traces recorded in somas in response to two-photon glutamate uncaging at spines (top, n=21 spines from 19 slices and 15 mice), filopodia (middle, n=22 filopodia from 20 slices and 16 mice), and filopodia in Mg²⁺ free ASCF with AMPA blocked (DNQX, 20 μM) (bottom, n=15 filopodia from 15 slices and 9 mice). Individual spine and filopodia traces in gray, population means in black.

FIG. 17d shows the population comparison of peak amplitudes of uncaging evoked responses from c. ****P<0.0001, Kruskal-Wallis test. Box plot represents median and IQR.

FIG. 17e shows an example two-photon image of a dendritic branch from a L5 V1 pyramidal neuron 41 filled with Alexa-594 and Fluo-4, showing spines and a filopodium. Changes in intracellular Ca²⁺ were measured via Fluo-4 signal at the filopodium and the parent dendrite in response to focal extracellular synaptic stimulation.

FIG. 17f shows a somatic voltage recording (top) and the corresponding changes in local Ca²⁺ (measured via Fluo-4 fluorescence; ΔF/F) at the tip of the filopodium shown in 17e (middle) and the parent dendritic branch shown in 17e (bottom) in response to focal extracellular synaptic stimulation in Mg²⁺-free aCSF with AMPA blocked (DNQX, 20 μM).

FIG. 17g shows a population analysis of the peak local Ca²⁺ signal in filopodia and their respective parent branches for synaptic stimulation successes (n=8 filopodia from 5 slices and 3 mice). **P<0.01, Wilcoxon signed-rank test. Box plot represents median and IQR.

FIG. 18a shows on Left: Two-photon z-stack of a V1 L5 pyramidal neuron filled with Alexa-488 via somatic patch pipette. Basal branch segment of interest indicated by box. Right: Magnified view of basal branch.

FIG. 18b shows (top) voltage response for the spine at lateral uncaging locations shown in a. (bottom) plot of lateral uncaging resolution. Continuous line is the Gaussian fit of the amplitudes of two-photon glutamate uncaging along lateral steps (circles).

FIG. 18c shows (top) voltage response for the spine at axial locations shown in 18a. Each voltage trace is an average of the voltage traces evoked at a specific axial step above and below of the spine. (bottom) plot of axial uncaging resolution (see 18b).

FIG. 18d shows a magnified view of a filopodium of a basal branch of a L5 pyramidal neuron. All uncaging experiments shown in e and f were performed in Mg²⁺ free ASCF with AMPA blocked (DNQX, 20 μM).

FIG. 18e shows the voltage response as in 18b, for the filopodium shown in 18d.

FIG. 18f shows the voltage response as in 18c, for the filopodium shown in 18d.

FIG. 19a shows superimposed traces of somatic voltage recordings (left) and corresponding changes in local Ca²⁺ (measured via Fluo-4 fluorescence; AF/F) at the parent dendritic branch (middle) and at the tip of the filopodium (right) in response to focal extracellular synaptic stimulation in Mg²⁺-free aCSF with AMPA blocked (via DNQX, 20 μM). All synaptic stimulation successes and failures for the filopodium in 17e are shown. Synaptic stimulation driven backpropagating action potential (bAP) also shown.

FIG. 19b shows the traces as in 19a with traces spaced apart. Grey dashed line indicates the onset of synaptic stimulation.

FIG. 20a shows a schematic of the induction pairing protocol.

FIG. 20b shows representative images (top) and somatic voltage traces (bottom) in response to two-photon glutamate uncaging test pulses at a filopodium before (left) and after (right) induction.

FIG. 20c shows Population averaged somatic voltage traces evoked by two-photon glutamate uncaging at filopodia (left, n=15 filopodia from 13 slices and 10 mice) and at spines (right, n=7 spines from 7 slices and 4 mice) before and after induction.

FIG. 20d shows Peak somatic uEPSP amplitude before and after induction in filopodia and spines. 3 different induction protocols were tested in filopodia: i—Pairing protocol (n=15 filopodia from 13 slices and 10 mice); ii—Somatic action potentials without any caged glutamate present (Post alone; n=7 filopodia from 7 slices and 6 mice); iii—Pairing protocol without somatic action potential (Pre alone; n=7 filopodia from 7 slices and 6 mice); ****P<0.0001, ns P>0.3.

FIG. 21 shows length of protrusions before and after induction in filopodia and spines. Three different induction protocols were tested in filopodia: i—Pairing protocol (n=15 filopodia from 13 slices and 10 mice); ii—Somatic action potentials without any caged glutamate present (Post alone; n=7 filopodia from 7 slices and 6 mice); iii—Glutamate uncaging without somatic action potential (Pre alone; n=7 filopodia from 7 slices and 6 mice); ns P>0.15. Wilcoxon signed-rank test. Box plot represents median and IQR with whiskers extending to the most extreme points not considered outliers.

FIG. 22a shows a schematic of the experiment. A control spine on a different branch than the branch of the test spine was always present. 40 and 90 repetitions of the pairing protocol were used for spines.

FIG. 22b shows the relative change of peak somatic uEPSP amplitude after pairing. P=0.5781 (40 repetitions, n=7 test and 7 control spines from 7 slices and 4 mice), P=0.9375 (90 repetitions, n=7 test and 7 control spines from 7 slices and 3 mice), Wilcoxon signed-rank test. Box plot represents median and IQR with whiskers extending to the 95% CI.

FIG. 22c shows the relative change of spine length after pairing. P=0.4688 (40 repetitions, n=7 test and 7 control spines from 7 slices and 4 mice), P=0.8125 (90 repetitions, n=7 test and 7 control spines from 7 slices and 3 mice), Wilcoxon signed-rank test. Box plot represents median and IQR with whiskers extending to the 95% CI.

FIG. 23a shows an example confocal image of a postnatal day (P) 13 Thy1-GFP+L5 pyramidal neuron dendritic segment after 4× expansion. Scale bar: 10 μm expanded/2.5 μm original.

FIG. 23b shows a fraction of dendritic protrusions classified as filopodia in P13 L5 PNs (n=371 dendritic protrusion, 18 dendritic branches, 3 mice). Box plot represents median and IQR with whiskers extending to 2σ.

FIG. 23c shows a fraction of total synapses in the three dendritic locations in P13 L5 neurons (n=397 synapses).

FIG. 23d shows (left) a box plot and individual data for signal intensity in Bassoon, NMDAR. and AMPAR channels for spines (n=236). (right) example four channel images of a representative spine. Box plot represents median and IQR with whiskers extending to the most extreme points not considered outliers.

FIG. 23e shows a fraction of dendritic protrusions as in 23b, but for filopodia (n=79).

FIG. 23f shows a fraction of dendritic protrusions as in 23b, but for shaft synapses (n=82). Example images show a shaft synapse that lacks AMPARs (top) and a shaft synapse that exhibits AMPARs (bottom).

FIG. 23g shows a comparison of dendritic protrusion types in P13 (n=371) and adult mice (n=2234).

FIG. 23h shows a comparison of synapse distribution in P13 (n=397) and adult mice (n=2188).

FIG. 24a shows a whole-cell patch clamp 2-photon image of a human cortical neuron.

FIG. 24b shows a confocal image of a human cortical neuron at an intermediate expansion 1.7×.

FIG. 24c shows 4 reslicing slice images and a side view of a human cortical neuron.

FIG. 24d shows an image of a human cortical neuron and a magnification in 24e.

FIG. 24f shows a human cortical neuron stained with Bassoon (left), GluN1 (middle) and GluA 1/2 (right).

FIG. 24g shows a human cortical neuron stained with Shank 3 (left), α-synuclein (middle) and PSD-95 (right).

FIG. 25a shows a 2-photon image of a spiny synapse.

FIG. 25b shows the AMPAR-uEPSP of s spiny synapse.

FIG. 25c shows the NMDAR-uEPSP of s spiny synapse.

FIG. 25d shows a confocal image of a spiny synapse.

FIG. 25e shows a spiny synapse stained with GluA1 & GluA2.

FIG. 25f shows a spiny synapse stained with GluN1.

FIG. 25g shows a neuron from Example cell 1.

FIG. 25h shows a 2-photon image of a neuron from Example cell 1.

FIG. 25i shows the glutamate uncaging result from a neuron from Example cell 1.

FIG. 25j shows a confocal image of a neuron from Example cell 1.

FIG. 25k shows a neuron from Example cell 1 stained with GluA1 & GluA2 (left) and GluN1 (right).

FIG. 25l shows a plot of AMPAR/NMDAR uEPSP vs. AMPAR/NMDAR intensity for a neuron from Example cell 1.

FIG. 25m shows a neuron from Example cell 2.

FIG. 25n shows a 2-photon image of a neuron from Example cell 2.

FIG. 25o shows a confocal image of a neuron from Example cell 2.

FIG. 25p shows the glutamate uncaging result from a neuron from Example cell 2.

FIG. 25q shows a neuron from Example cell 2 stained with GluA1 & GluA2 (left) and GluN1 (right).

FIG. 25r shows a plot of AMPAR/NMDAR uEPSP vs. AMPAR/NMDAR intensity for a neuron from Example cell 2.

FIG. 26a shows a z-projection of branch on a synaptic protein.

FIG. 26b shows z-slices containing the spine of interest and their AMPAR/NMDAR intensity.

FIG. 26c shows z-slices not containing the spine of interest and their AMPAR/NMDAR intensity.

FIG. 26d shows a plot of AMPAR/NMDAR uEPSP vs. AMPAR/NMDAR intensity.

FIG. 26e shows a plot of the Pearson correlation coefficient.

FIG. 27a shows AMPAR and NMDAR EPSP.

FIG. 27b shows AMPAR/NMDAR uEPSP in mouse RSC (left), mouse V1 (middle) and human TL (right) spines.

### DETAILED DESCRIPTION OF THE INVENTION

Animal models of human diseases are key in understanding the disease mechanisms and probing potential therapies. However, central nervous system (CNS) disorders pose significant challenges in creating useful models due the divergence of human brain organization and function. Human neurological disorders e.g., autism, dementia and schizophrenia are complex and hard to model in animals. Less than 5% of the drugs that have been proven beneficial in animal models for neurological diseases are eventually approved for use in humans. Since animal models are not predictive of human diseases, patients become the end-point experimental model for evaluating the biology, which is an expensive and time-consuming proposition. Patch2MAP allows the direct investigation of the human brain and can inform better drug developing strategies.

Alzheimer's disease (AD) is a common neurodegenerative disease of the elderly, afflicting 6 million people in the United States alone. Remarkably few treatments have been developed and none have shown clinically significant evidence of slowing or preventing AD in humans. Animal models are generally non-predictive of human AD. Disclosed herein are methods of measuring synaptic dysfunction in AD patients that is directly linked to cognitive decline.

One of the earliest hallmarks of AD-related cognitive decline is the disruption of neuronal connectivity, observable as net synapse loss. Changes in synaptic density are even found in patients with mild cognitive impairment (MCI), a prodromal state of AD. Human genetic studies suggest that the majority of identified AD risk genes are expressed in microglia rather than neurons. Given the well-established role of microglia in sculpting synaptic function during development, microglia are likely to have a leading role in the pathophysiology of AD synaptic dysfunction. Studies from animal models indicate a two-faced role of microglia during disease progression (where they are protective of synapses in the early stages of disease and catastrophic in later stages). All current super-resolution methods require exogenous protein expression to identify the morphology of the cells of interest, such as genetically modified animals or viral transduction. This limits the applicability of these techniques in terms of cell types and species, which is particularly relevant for the human brain. The present disclosure couples patch-clamp electrophysiology and eMAP to achieve super-resolution morphological and proteomic imaging in any cell type and species without exogenous expression. Combining the two techniques required technical innovation that resulted in an unexpected outcome. This method allows super-resolution cell-delineated proteomics in the human brain.

Super-resolution microscopy enables the investigation of proteins with nanoscopic resolution. Among them synthetic gel-based super-resolution imaging techniques, which include eMAP and E×M, achieve ultrastructural imaging by coupling physical expansion of tissue with diffraction-limited microscopy. eMAP further provides the opportunity of highly multiplexed proteomic analysis, as the processed tissue is amenable to multiple rounds of staining and destaining. However, all current super-resolution methods require exogenous protein expression to identify the morphology of cells of interest, such as genetically modified animals or viral transduction. This limits the applicability of these techniques in terms of cell types and species. This limitation is particularly relevant for human neurons. Although super-resolution microscopy can be used to investigate protein distribution in the human brain, the lack of single cell morphological labeling prevents accurate assignment of a given protein of interest to cells and their subcellular compartments. This precludes systematic analysis of protein distribution in native human tissue. Patch2MAP was developed, a method that combines the advantages of patch-clamp electrophysiology with eMAP for concomitant super-resolution structural and proteomic investigation of single cells in brain tissue from any species.

Patch2MAP, a technique that combines patch-clamp physiology with super-resolution structural and protein imaging in human brain tissue. Patch2MAP works by collecting physiological measurements of the cells of interest (including subcellular measurements, like synaptic function), filling them with a dye, and then physically expanding the tissue for imaging beyond the diffraction limit. It allows multiple rounds of protein staining and destaining and is well suited for concomitant functional, structural and protein investigation of single synapses and their relation to microglia.

Recent developments in super-resolution microscopy have revolutionized the study of cell biology. However, in dense tissues, exogenous protein expression is required for single cell morphological contrast. This presents challenges in the nervous system, where many cell types and species of interest are not amenable to genetic modification and intricate anatomical specializations make cellular identification difficult. Provided herein is a method for full morphological labeling of individual neurons from any species or cell type for subsequent cell-delineated protein analysis. This method, which combines patch-clamp electrophysiology with epitope-preserving magnified analysis of proteome (eMAP), additionally allows for correlation of physiological properties with subcellular protein expression. Patch2MAP shows that functional AMPA-to-NMDA receptor ratios correspond to the expression level of these receptors at single spiny synapses in human cortical pyramidal neurons. Patch2MAP thus allows the concomitant investigation of the anatomical and proteomic ultrastructure of human neurons, opening new avenues for direct molecular investigation of the human brain in health and disease.

Patch clamp technology was developed four decades ago and has been used to study the biophysical properties of cells and their components. It measures the flow of ionic currents across cell membranes. Patch-clamp uses a hollow glass tube known as a patch pipette filled with an electrolyte solution and a metal recording electrode wire connected to an amplifier. The patch pipette is brought into contact with the membrane of a target cell. Electrical activity of the cell is measured, and the cell is identified by its electrical activity based on the characteristic ion channels expressed in its membrane.

Patch-clamp technology can be used to measure electrical activity of cells that are viably maintained in acute tissue slices, cultures or cell lines. Electrophysiologists have previously applied patch-clamp measurements directly in animals to analyze brain and/or central nervous system function. One aspect of the disclosure is a method of evaluating a disease, such as autism, dementia, or schizophrenia, by analyzing a brain tissue sample using Patch2MAP. Another aspect of the disclosure is a method of monitoring treatment of a disease state from a brain tissue sample using Patch2MAP. Another aspect of the disclosure is using Patch2MAP to measure the effects of a therapeutic treatment on human neural cells derived from a brain tissue sample. Another aspect of the disclosure is a kit for analyzing a biopsy brain tissue sample of neural tissue to detect a neural disease or to monitor treatment. Another aspect of the disclosure is correlating the protein content of subcellular structures with patch-clamp measurements of brain tissue sample by the Patch2MAP technology.

The present disclosure provides a method for processing cells from a neural tissue sample to measure subcellular localization of proteins. The processing steps maintain the structural integrity of the cells following the patch-clamp measurement. The processing steps include fixing the cells, microdissection, and tissue slicing, and permits multiple rounds of destaining and restaining for more proteins.

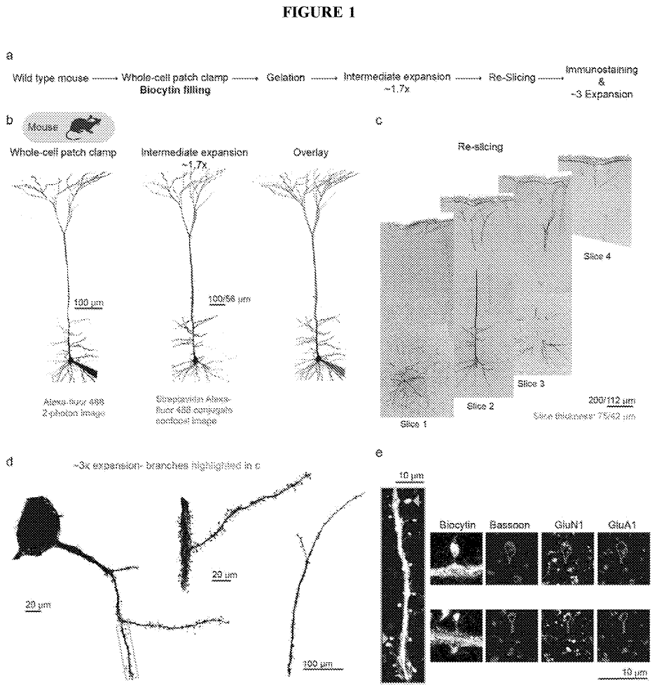

To develop a system for morphological labeling of individual cells amenable to subsequent proteomic analysis, the well-known approach for full anatomical reconstruction via the biocytin-streptavidin complex was used. This method consists of acquiring a whole-cell patch-clamp recording of a single cell, filling the patched neuron with biocytin, fixing the brain slice, and utilizing the very strong non-covalent bond between streptavidin and biotin to introduce a fluorophore selectively to the filled cell. This approach allows for high density filling of neurons and morphological reconstruction of fine processes including dendritic spines and axons. Cortical pyramidal neurons from acute brain slices of adult mice were used as the model. To achieve super-resolution proteomic imaging of biocytin-filled cells, the original eMAP protocol (FIG. 1a) was modified. Hydrogel tissue hybridization may alter the biotin-streptavidin-fluorophore complex. Indeed, after the tissue-gel hybridization the biocytin distribution was not able to be recovered and reconstruct the neuron, irrespective of when the streptavidin-Alexa Fluor 488 was introduced (before or after the tissue-gel hybridization). Furthermore, the morphology of the neuron was not able to be recovered even after using antibodies that target streptavidin. To maintain the streptavidin-fluorophore signal, an additional step of fixation before gelation was included. The tissue was washed thoroughly to remove any excess formaldehyde and then incubated in a hydrogel monomer solution containing 30% acrylamide, 10% sodium acrylate, 0.1% bis-acrylamide, and 0.03% VA-044. This process allowed recovery of the biocytin intracellular distribution and visualize the morphology of the neuron in the tissue-gel hybrid at intermediate expansion ratio (FIG. 1b). Using a vibratome, the original 300 μm thick slice was resliced-typical thickness for slice physiology-in thinner slices (FIG. 1c) and incubated the resulting slices in a denaturation solution containing [6% SDS (w/v), 50 mM sodium sulfite, and 0.02% sodium azide (w/v) in PBS. Commercially available antibodies were then used to target synaptic proteins and subsequently expanded the tissue-gel in deionized (DI) water to visualize the ultrastructure of synapses (FIG. 1d, and 1e)

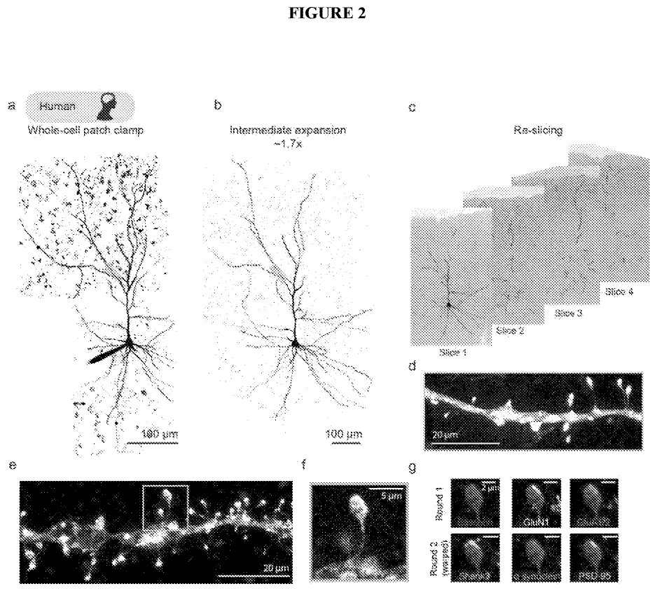

To evaluate Patch2MAP in human neurons, fresh human cortex was acquired from neurosurgical patients. Blocks of cortical tissue were maintained in slicing aCSF, microdissected, and then sliced as was done for mouse cortex. Whole cell patch-clamp recordings of human neurons were acquired and the same steps were used as described in mice (FIGS. 2a, 2b, and 2c). Patch2MAP effectively produced bright and highly specific morphological and proteomic signals in human neurons and synapses (FIGS. 2d, 2e, 2f, and 2g). Furthermore, eMAP-processed human tissue gel was shown to endure destaining and restaining of proteins (FIG. 2g), allowing for the investigation of multiple proteins in each ultrastructural compartment. Thus, Patch2MAP can be used in human tissue and enable the concomitant ultrastructural investigation of human neuron anatomy and proteomics.

Patch2MAP's integrated nature was used to directly test how physiological properties correspond to subcellular protein expression. AMPA and NMDA receptors at single spiny synapses in human cortical pyramidal neurons were evaluated, known for their unique role in synaptic transmission and plasticity. Despite more than 40 years of rigorous investigation of these receptors it was still unclear how, or even if, the receptor protein number for a given synapse relates to its functional synaptic strength.

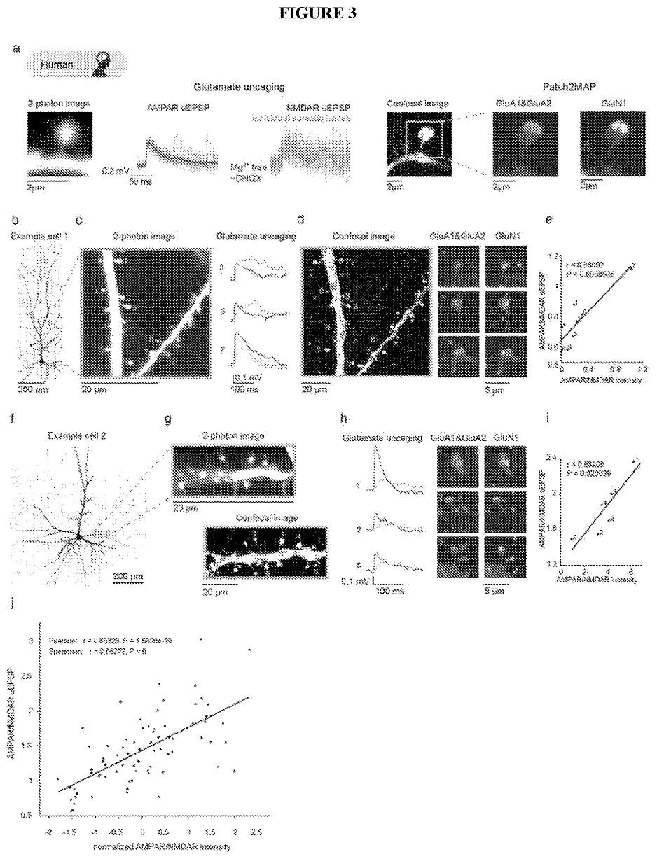

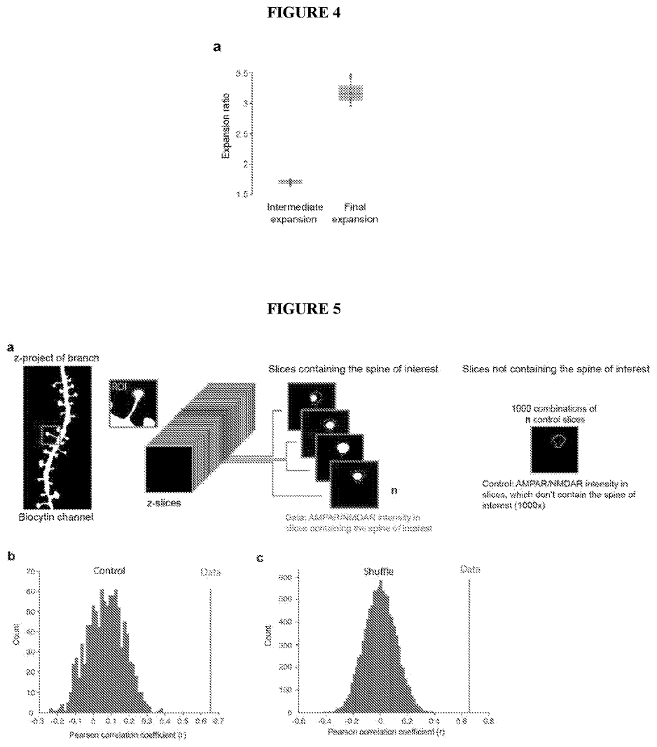

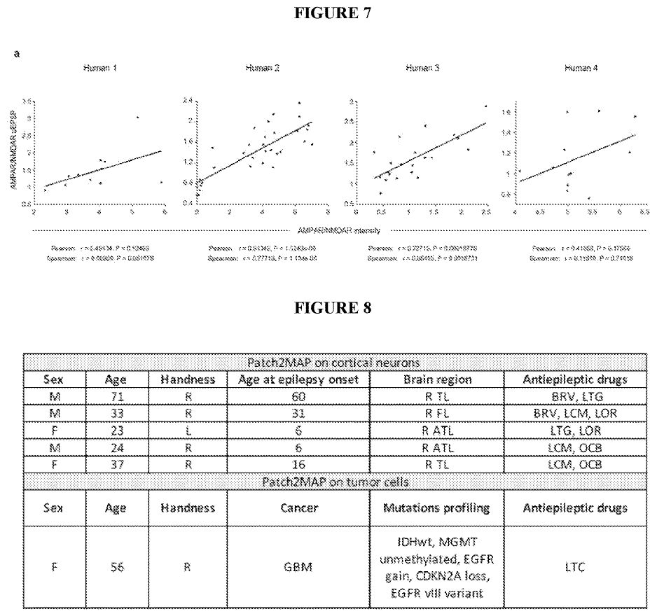

Whole cell patch-clamp recording and rapid two-photon glutamate uncaging were used to measure functional AMPA-to-NMDA receptor ratios at individual spiny synapses. The response of a given spine to glutamate uncaging heavily relies on the uncaging laser power, as well as the uncaging location relative to the spine of interest and the local delivery of the caged glutamate compound. Additionally, spine neck resistance and dendritic filtering also affect the amplitude of synaptic responses recorded in the soma. To account for these potential confounds, the AMPA-to-NMDA receptor ratio was measured (AMPA:NMDA) instead of relying on AMPA amplitude. AMPA:NMDA provides a measurement that is largely independent from the uncaging parameters and dendritic filtering. To do this, individual spines were activated at a given branch with glutamate uncaging under control conditions in current-clamp mode to produce approximately physiologically-sized unitary uEPSPs (FIG. 3a). The slice was perfused with DNQX and 0.0 mM Mg²⁺ aCSF and repeated the uncaging was repeated at the same spines (FIGS. 3a, 3c, 3g, and 3h). This created a ratio of mostly AMPAR-mediated uEPSP amplitude to mostly NMDAR-mediated uEPSP amplitude. After the recording, the tissue was processed for subsequent super-resolution imaging, staining the tissue gel hybrid for GluA1 and GluA2 subunits of AMPAR and GluN1 subunit of NMDAR receptors. After ~3× expansion of the tissue (FIG. 4) the dendritic branch containing our spines of interest was identified and the AMPA:NMDA signal intensity was computed for every spine that was activated by glutamate uncaging (FIGS. 3a, 3d, 3h, and FIG. 5). The functional AMPA:NMDA correlates strongly and significantly with the protein AMPA:NMDA (FIG. 3j, FIGS. 5, 6 and 7; n=76 spines, 11 cells, 4 human patients; Pearson correlation coefficient=0.65, P-value=1.6e-10). Thus, the protein content measured with Patch2MAP predicts synaptic transmission strength in human cortical neurons. Furthermore, such a result validates that the signal intensity of labeled proteins reliably predicts protein content.

Glioblastoma (GBM) is the most common primary brain tumor in adults. It exhibits poor prognosis even with maximal therapy 27. Glioblastomas synaptically integrate into surrounding neuronal networks by forming electrochemical synapses. Neuronal activity promotes tumor growth and tumors themselves can remodel the activity and function of neural circuits. However, GBM disease mechanisms are not well understood as glioblastomas exhibit a profuse cellular heterogeneity in their composition and each cell's contributions cannot be studied in isolation. Animal models capture only a subset of glioblastoma manifestations in the human brain. Patch2MAP can elucidate both the function and molecular composition of human GBM on a single cell level embedded in the complex native tumor environment.

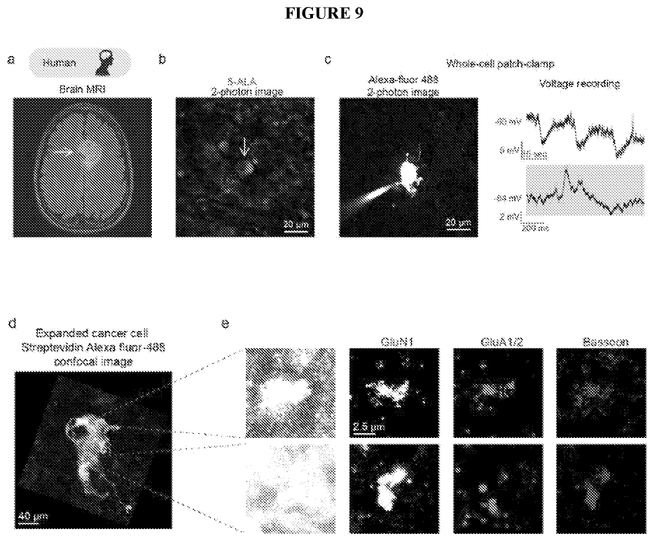

In one embodiment, freshly resected cortical tissue was acquired from a patient with glioblastoma (FIG. 9a) undergoing surgical treatment. The patient was administered 5-ALA preoperatively, which is metabolized to fluorescent protoporphyrin IX (PpIX) specifically in tumor cells and allows intra-operative delineation of tumor borders from surrounding brain. A 5-ALA labeled tissue block arising from the 144 upper border of the tumor was prepared for Patch2MAP analysis. Tumor cells were identified using two-photon imaging and exhibited 5-ALA-mediated fluorescence, which was nucleus excluded (FIG. 9b). Under visual guidance, patch-clamp whole-cell current-clamp recording was obtained from the tumor cell (FIG. 9c). The voltage recording revealed spontaneous slow oscillatory activity (FIG. 9c), as well as faster depolarizations resembling synaptically-driven electrical activity (FIG. 4c inset). Neuron-to-glioma synapses are present in human GBM with transcriptomic and pharmacologic experiments arguing for the existence of AMPARs. To directly visualize synaptic receptors on human resected GBM tissue, the patched and filled tumor cell was processed through Patch2MAP and stained for AMPARs (GluA1&GluA2), NMDARs (GluN1) and Bassoon. During super-resolution confocal imaging, synapses on the surface of the tumor cell of interest were observed that contained both AMPAR and NMDAR receptors (FIGS. 9d, 9e). Calcium influx plays a central role in the growth and invasiveness of glioblastoma, orchestrating processes which normally take place during the development of the nervous system 10. Calcium permeable AMPARs of neuron-to-glioma synapses facilitate this calcium influx in tumor. Patch2MAP demonstrates that neuron-to-glioma synapses have both AMPAR and NMDAR receptors, indicating how neuronal activity can induce calcium influx in the tumor cells and promote tumor growth.

Patch2MAP can also be used in human tissue acquired from rapid autopsy of AD patients to assess synapse structure, function, and protein nanodomains across disease stages and correlate these properties with proximity to Aβ plaques and intracellular neurofibrillary tangles. Multiple synaptic proteins needed for normal synapse function interact with Aβ and tau and form nanodomains, including receptors (AMPARs, NMDARs), signaling molecules (FYN, Shank 3)29 and scaffolding proteins (PSD 95, Bassoon). Disruption of protein organization at the nanoscale may account for early synaptic dysfunction before gross anatomical changes are apparent. This technique can elucidate the link between synaptic dysfunction and aberrant protein expression as a function of AD progression.

Microglia protect synaptic function in early stages of AD by sealing extracellular Aβ, but in later stages, microglia drive synaptic loss by inflammation and engulfment of synapses. Patch2MAP can help determine if synaptic dysfunction is less prominent to microglia-sealed Aβ plaques (vs-unsealed plaques) in early stages of the disease and more prominent adjacent to microglia in late stages of the disease. To visualize microglia, a microglia specific antibody (Iba1) is used. To access whether different activation states of microglia mediate the opposing effects in synapses, microglial proteins (C1q complement, CX3CR1, Trem2, Tyrobp, ApoE) can be stained in a series of multiple rounds. Super-resolution imaging of microglia's and synapses' structure and protein content dissect the microglia-synapse interplay at the nanoscale. Different modes of microglial function can be induced either by introducing Aβ oligomers or inflammatory cytokines to organotypic cultures. Gross synaptic changes can be followed dynamically, by imaging genetically targeted human neurons with viral vectors, while markers of microglia activation states and subsynaptic protein expression profiles identified in using Patch2MAP will be evaluated with super-resolution imaging. In some embodiments, investigations can occur using 2 cm×2 cm×2 cm cubes containing cortical and hippocampal tissue obtained from a fresh autopsy of an AD patient are placed in cold artificial cerebrospinal fluid (aCSF) for electrophysiology recordings and super-resolution imaging (Patch2MAP).

Newly generated excitatory synapses in the mammalian cortex lack sufficient AMPA-type glutamate receptors to mediate neurotransmission, resulting in functionally silent synapses that require activity-dependent plasticity to mature. Silent synapses are abundant in early development, where they mediate circuit formation and refinement, but they are thought to be scarce in adulthood. However, adults retain a capacity for neural plasticity and flexible learning that suggests that the formation of new connections is still prevalent. Super-resolution proteomic imaging was used to visualize synaptic proteins at 2,234 synapses from layer 5 pyramidal neurons in the primary visual cortex of adult mice. Surprisingly, ~25% of these synapses lack AMPA receptors. These putative silent synapses were located at the tips of thin dendritic protrusions, known as filopodia, which were more abundant by an order of magnitude than previously believed (compromising ~30% of all dendritic protrusions). Physiological experiments revealed that filopodia do indeed lack AMPAR-mediated transmission, but they exhibit NMDAR-mediated synaptic transmission. Functionally silent synapses on filopodia can be unsilenced via Hebbian plasticity, recruiting new active connections into a neuron's input matrix. These results challenge the model that functional connectivity is largely fixed in the adult cortex and demonstrate a new mechanism for flexible control of synaptic wiring that expands the learning capabilities of the mature brain.

Synaptic plasticity is implemented by the strengthening or weakening of neural connections as well as the formation of wholly new synapses or elimination of existing ones. In the adult mammalian brain, plasticity is thought to manifest mainly via scalar changes in synaptic strength of existing connections. Silent synapses are prevalent in developing cortex, where they facilitate a highly flexible connectivity matrix. Silent synapses may be prevalent in mature brains, where they contribute to neural plasticity and learning.

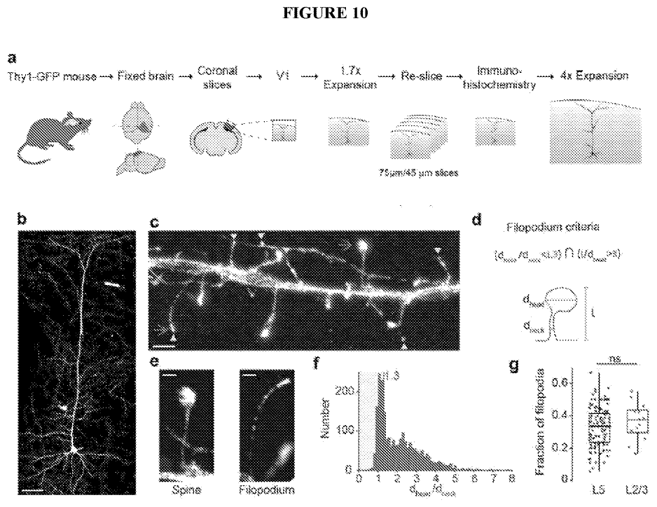

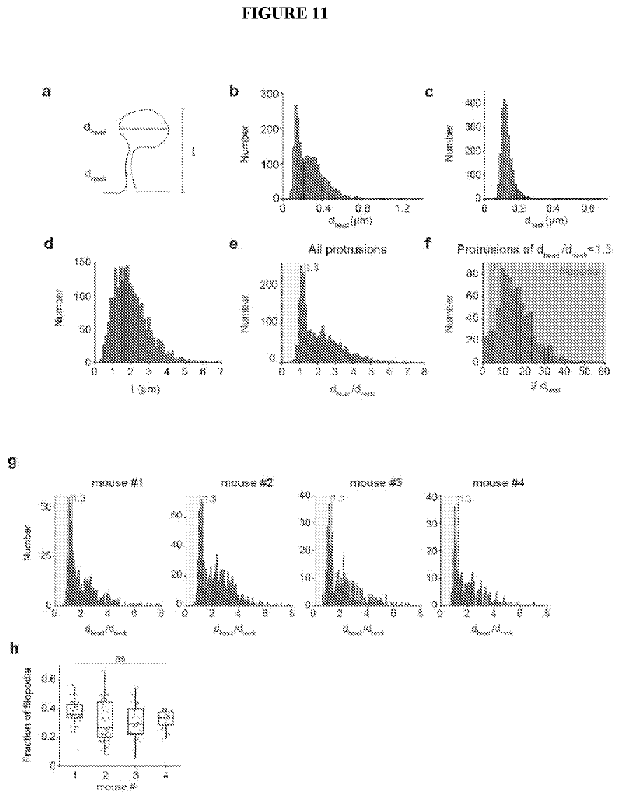

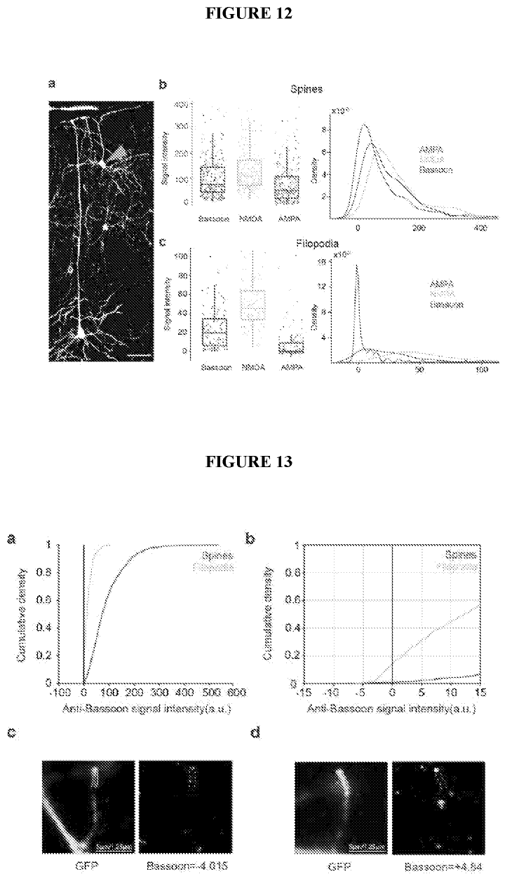

Epitope preserving Magnified Analysis of the Proteome (eMAP) was performed to acquire super-resolution images of dendritic protrusions along with their synaptic AMPA and NMDA receptor protein content in layer 5 (L5) pyramidal neurons (PNs) from the primary visual cortex (V1) of adult mice. Brains were collected from 4 adult Thy1-GFP-M+ mice, which feature sparsely labeled cortical PNs, mostly from L5 (FIG. 10a). Synapses were sampled across the full cortical thickness of V1, randomly selecting in-plane dendritic segments from L5 PNs for imaging (FIGS. 10b, 10c). Within each ~20 μm long dendritic segment, all protrusions were annotated. 123 dendritic segments were imaged for a total of 2,234 dendritic protrusions in L5 PNs. Filopodia, classically defined as protrusions lacking distinct heads (FIGS. 10d, 10e, 11), accounted for ~30% of the total number of dendritic protrusions imaged (FIGS. 10f, 10g). This corresponds to an order of magnitude higher prevalence in adults than previously reported. The high percentage of filopodia in adult L5 neurons is not a unique property of these cells, as a similar percentage of filopodia were observed in L2/3 neurons expressing virally transduced GFP (FIGS. 10g, 12).

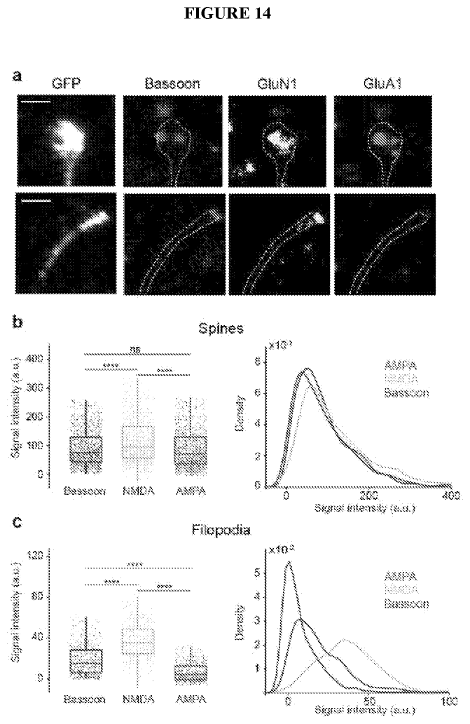

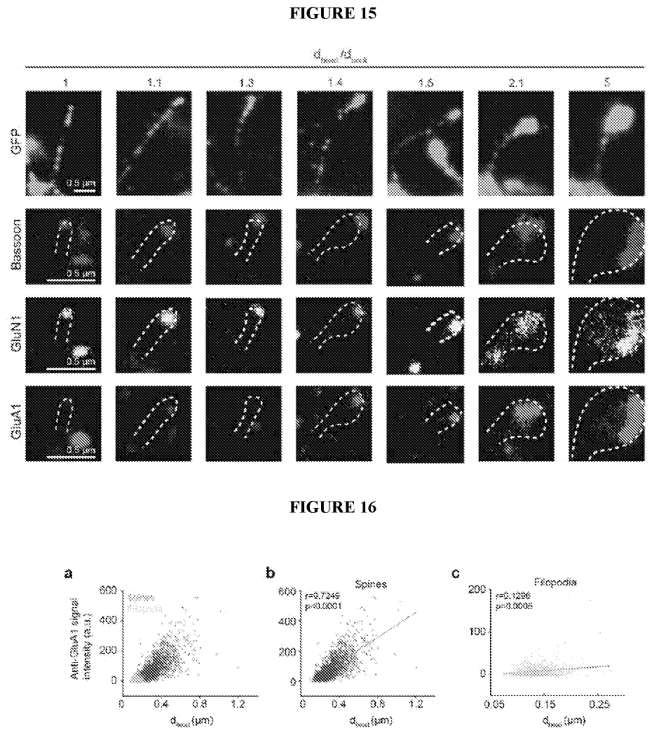

Filopodia were evaluated for synaptic receptors necessary for neurotransmission. Antibodies against Bassoon (a presynaptic protein involved in vesicle clustering) were used to visualize presynaptic compartments located adjacent to GFP-expressing postsynaptic structures of interest. Almost all spines (99.14%) and the majority of filopodia (85.75%) exhibited a Bassoon-defined presynaptic partner (FIG. 13). Antibodies for GluA1 and GluN1 were used to label AMPA and NMDA receptors, respectively. As expected, dendritic spines contained both AMPA and NMDA receptors (FIGS. 14a, 14b, 15); AMPAR signal increased as a function of spine head size, consistent with previous studies (FIG. 16). Filopodia, however, lacked AMPAR signals while exhibiting robust NMDAR signals (FIGS. 12, 14a, 14c, 15, 16). Thus, using super-resolution protein imaging, filopodia appear as GluA1 immune-negative, GluN1 immune-positive protrusions that contact Bassoon immune-positive presynaptic membranes.

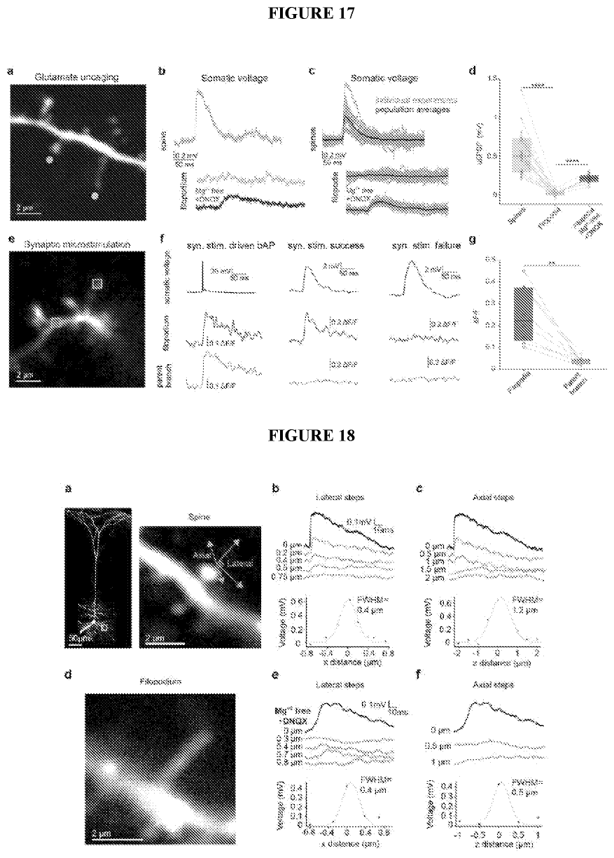

Filopodia may contain synapses that are electrically silent at resting membrane potential, due to voltage-dependent block of NMDARs by Mg²⁺. Silent synapses have classically been defined as a mismatch between the number of responsive synapses at resting membrane potential and the number of responsive synapses at depolarized membrane potential. Such synapses contain NMDARs but no or very few AMPARs. eMAP enables exceptional preservation of antigenicity; however, as with any other immunohistochemical technique, low levels of protein expression may be difficult to detect. By combining whole cell patch clamp electrophysiology with two-photon glutamate uncaging and imaging, filopodia were tested to see if they represent functionally silent synapses in brain slices of adult mouse primary visual cortex. L5 pyramidal neurons were filled with a structural dye (Alexa 488) to target two-photon glutamate uncaging onto identified postsynaptic structures. Only a subset of filopodia were observed under optimized 2-photon imaging due to the resolution limit and to the optical aberration associated with other nearby brightly labeled structures like large spines and parent dendrites. Uncaging laser power was calibrated using spines near (<10 μm) targeted filopodia, such that spine uncaging elicited large (0.2-1.2 mV) somatic EPSPs (FIGS. 17a, 17b, 18). All spines tested (21/21) exhibited synaptic responses to glutamate uncaging. In sharp contrast, none of the filopodia (22/22) responded to glutamate uncaging (FIGS. 17b, 17c, 17d). The lack of somatic response could indicate a lack of AMPA receptors, or to filtering across the long, potentially high resistance filopodia neck. To investigate these possibilities, Mg²⁺-free aCSF was used to relieve the voltage-dependence of NMDARs while also blocking AMPARs (with 20 μM DNQX). Under these conditions, NMDAR-mediated EPSPs were observed in response to glutamate uncaging at filopodia (FIGS. 17b, 17c, 17d). These results indicate that neck resistance filtering is not responsible for the lack of AMPA-mediated responses at filopodia. Instead, this physiological data demonstrates that filopodia lack AMPAR-mediated transmission but do exhibit NMDAR-mediated transmission, consistent with disclosed proteomic imaging results.

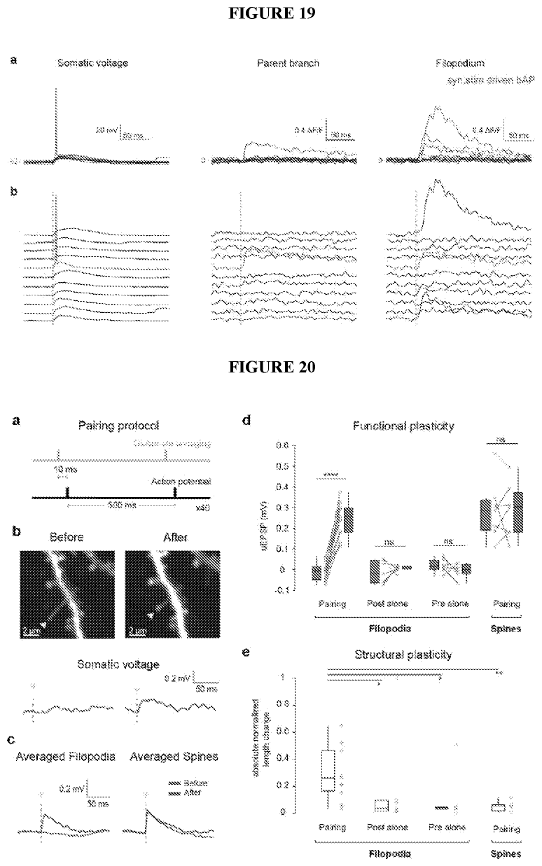

The majority of filopodia exhibited Bassoon-defined presynaptic partners, but uncaging experiments did not establish if filopodia-associated presynaptic structures are vesicle release competent. Thus, the presence of synaptically-evoked local Ca²⁺ transients was determined in filopodia in response to electrical microstimulation of nearby axons. L5 PNs were filled with a structural dye (Alexa 594) and a calcium indicator (Fluo-4) and placed a bipolar theta glass microelectrode ~10 μm from a dendritic branch with an identified filopodium. Microstimulation was conducted in Mg²⁺-free aCSF with AMPARs blocked (20 μM DNQX). Synaptic transmission was measured local Ca²⁺ transients (via Fluo-4) in filopodia (FIGS. 17e, 17f, 19). Large local Ca²⁺ signals were observed in response to microstimulation in the tip of filopodia that were not detectable in the parent dendrite, indicating that their associated presynaptic structures are indeed release competent. Successful transmitter release was distinguished from failure of release, and these events were clearly distinct from widespread electrical events like backpropagating action potentials (FIGS. 17e, 17g). Combined with glutamate uncaging and super-resolution proteomic imaging experiments, these results demonstrate that filopodia are a structural substrate for silent synapses in the adult brain.

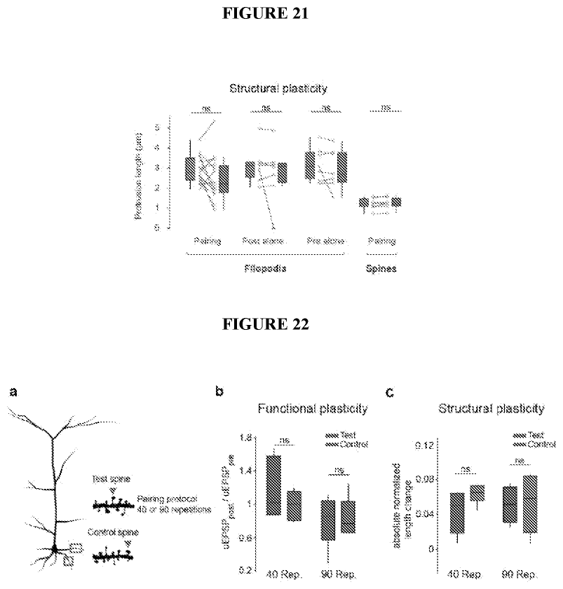

The activity-dependent conversion of silent to functional synapses plays a key role in developmental plasticity. However, the contribution of silent synapses to adult cortical plasticity has not been determined due to low silent synapse prevalence in adult brain. Disclosed herein are studies regarding whether adult silent synapses at filopodia can be converted into active (i.e. not silent) synapses. Multiple studies have previously provided evidence for unsilencing silent synapses at the population level in the developing brain. Morphologically identifying silent synapses allows for determining whether they could be “unsilenced” at the individual level in the adult brain. A spike timing-dependent plasticity (STDP) protocol was used where presynaptic activity was mimicked by glutamate uncaging at filopodia and closely followed (10 ms later) by current injection in the soma of the postsynaptic neuron to produce a single action potential (FIG. 20a). After induction of the plasticity protocol, the length of filopodia changed (9/15 decreased by 36±6% while 6/15 increased by 18±7%, FIG. 21). Induction resulted in the appearance of AMPAR-mediated synaptic responses at filopodia on the timescale of minutes (FIGS. 20b, 20c, 20d). The structural and functional changes in filopodia were observed only when pre- and post-synaptic activity was paired (FIGS. 20d, 20e). This protocol did not induce either structural or functional plasticity at conventional spiny synapses 30 (FIGS. 20c, 20d, 21). This is in contrast with previous reports, where the same protocol plasticized spines in the juvenile brain. Plasticity was not induced at spines even when the number of EPSP-AP pairings were more than doubled (FIG. 22). Thus, an STDP plasticity protocol is insufficient to induce synaptic plasticity in spines of adult animals but is sufficient to unsilence silent synapses, demonstrating a hierarchy of plasticity thresholds for different synaptic classes in the adult mammalian cortex.

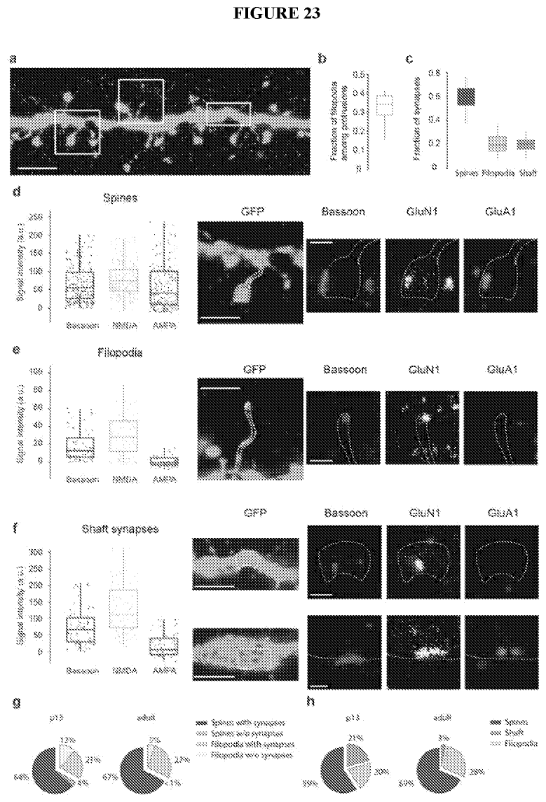

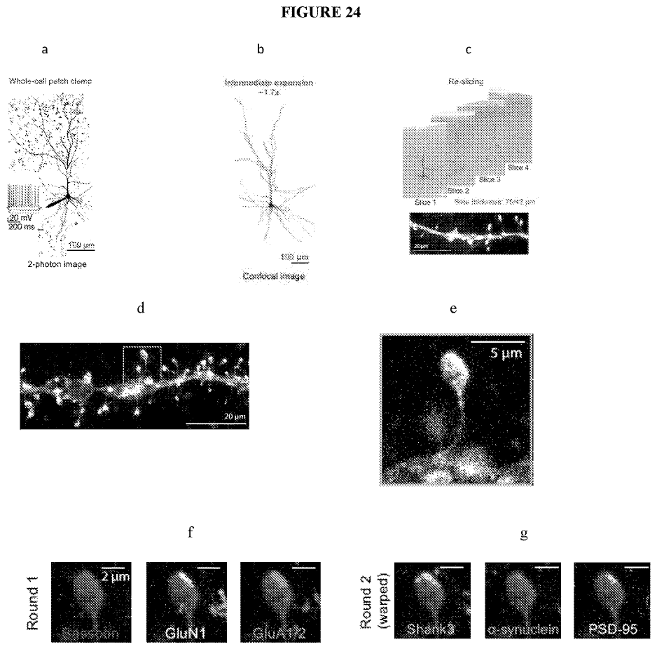

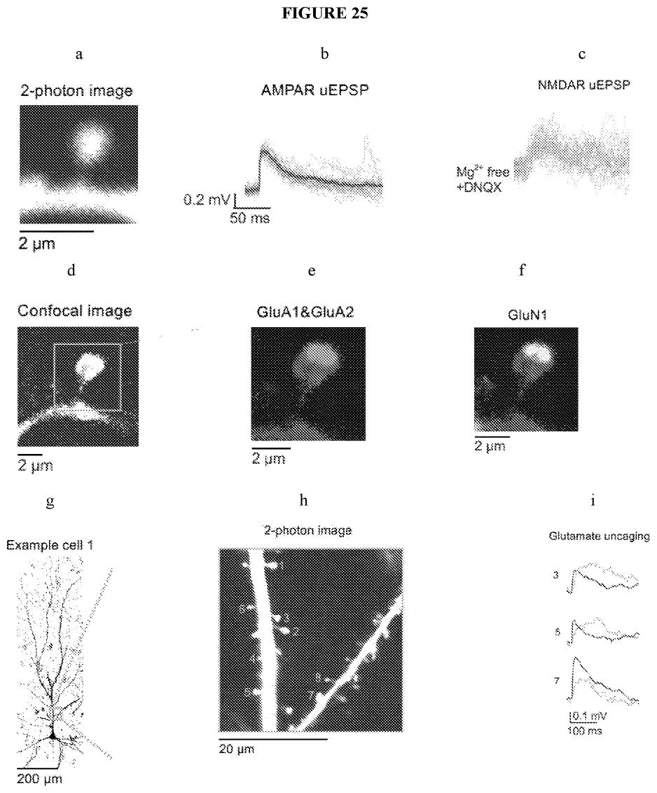

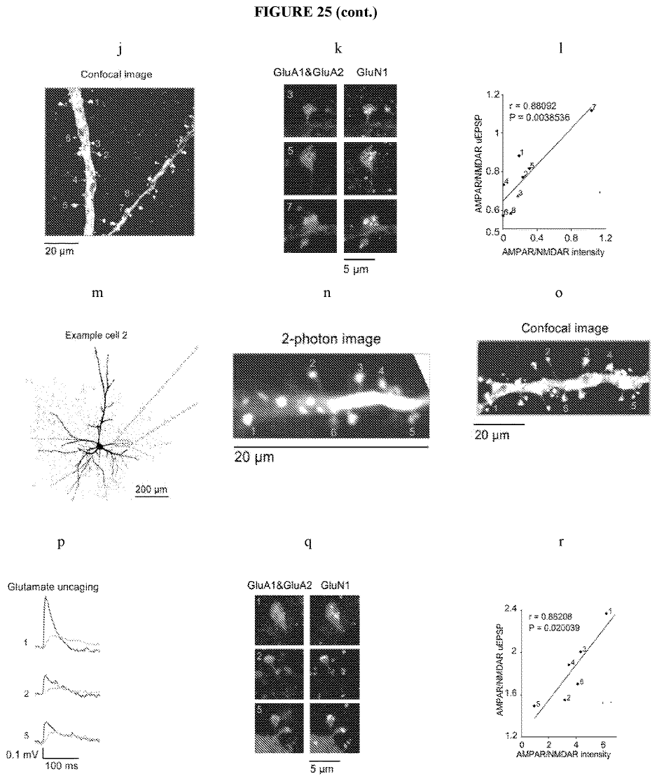

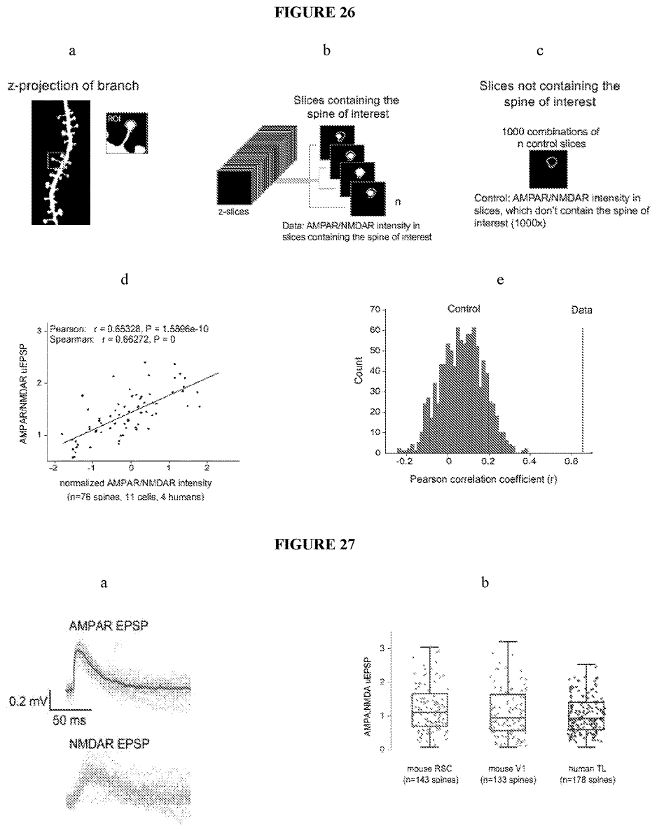

The super-resolution imaging technique applied here allows for resolution of structures well below the diffraction limit. Although eMAP retains original micro-architecture without distortion, to compare the approach with the existent synapse morphology literature in juveniles, eMAP was performed in 3 Thy-1-GFP-M+ mice at postnatal day 13 (PN13, (FIG. 23). In accordance with previous reports16, our experiments revealed a high percentage of shaft synapses in PN13 (FIG. 23). Furthermore, our morphological measurements of dendritic protrusions in adults are consistent with prior electron microscopy measurements (FIG. 11).

eMAP experiments revealed small postsynaptic structural details, including filopodia, that are usually hidden in the haze of fluorescence around dendrites and large spines in conventional light microscopy. The combination of physical enlargement of the tissue, single cell morphological contrast, and optical clearing, allowed for nanoscopic investigation of synaptic structures. In addition to the unexpectedly high number of filopodia observed (~30% of dendritic protrusions in mouse V1 L5 and L2/3 PNs), a high number of functionally silent synapses were identified in the adult cortex. Disclosed herein is data directly linking dendritic filopodia to silent synapses, showing that they represent a substantial and novel reservoir for adult cortical plasticity. Silent synapses have a different threshold for plasticity than non-silent synapses, which is consistent with theoretical models of flexible and robust memory. Specifically, memory formation requires a balance between flexibility and stability: flexibility establishes memory acquisition and stability ensures memory retention. Mature synapses at spines stably store acquired information, while silent synapses at filopodia mediate the rapid acquisition of new information.

Disclosed herein is a new method to perform super-resolution proteomic microscopy at identified subcellular structures by coupling patch-clamp electrophysiology with eMAP. This approach provides a generally applicable framework to visualize the subcellular morphology and protein content of any neuron without exogenous protein expression. Moreover, biophysical properties to proteomic profiles of synapses are linked and signal intensity of labeled proteins measured through Patch2MAP can be reliably used to infer the protein concentration. Disclosed herein are methods using resected human GBM tissue to visualize the molecular nanostructure of neuron-to-glioma synapses. The present disclosure enables the ultrastructural cell-delineated proteomic analysis of any neuron of any species, including human, and paves the way for further studies in brain health and disease.

Animal models of human diseases are key in understanding the disease mechanisms and probing potential therapies. However, central nervous system (CNS) disorders pose significant challenges in creating useful models due the divergence of human brain organization and function. Patch2MAP allows the direct investigation of the human brain and has the potential to change what is considered a clinical sample and how it can contribute to understanding the mechanism of neurological diseases.

Disclosed herein are methods of producing patch-clamp measurements from neural cells derived from neural tissue samples and how to detect and measure subcellular localization of proteins from the same neural cells.

### Embodiments

1. A method of analyzing neurons in a subject with normal neurologic function or a subject having neurological and/or neuropsychiatric disease, comprising
a. obtaining a brain tissue sample from the subject, wherein the brain tissue sample comprises viable neuronal tissue;
b. patch-clamping one or more neurons in the viable neuronal tissue to record their individual electrical activity, wherein the one or more neurons are alive during patch-clamping;
c. filling the patch-clamped neurons with a dye to identify morphological features of the one or more neurons in the neuronal tissue; and
d. processing the brain tissue sample to physically expand it; and
e. treating the brain tissue sample with an antibody that enables detection of a protein in the one or more neurons.
2. The method of embodiment 1, wherein the neurological and/or neuropsychiatric disease is selected from attention-deficit/hyperactivity disorder (ADHD), autism spectrum disorder (ASD), communication disorders, intellectual developmental disorder, intellectual disability, learning disorder, disruptive mood dysregulation disorder, dementias (Alzheimer's disease, vascular dementia, Lewy body dementia, frontotemporal lobar degeneration), brain cancer, glioblastoma, epilepsy, movement disorders (Parkinson's disease, Huntington's disease, multiple system atrophy, progressive supranuclear palsy, restless leg syndrome, tremor, Tourette syndrome), stroke, Transient Ischemic Attack, functional neurological disorders, anxiety disorders, mood disorders, psychotic disorders, eating disorders, impulse control and addiction disorders, and personality disorders.
3. The method of embodiment 1, wherein the neurological and/or neuropsychiatric disease is glioblastoma or Alzheimer's disease.
4. The method of embodiment 1, wherein the dye comprises biotin or biotin-based dyes and biotin binding proteins (e.g. fluorophore-labeled streptavidin).
5. The method of embodiment 1, further comprising treating the tissue with formaldehyde and/or a hydrogel.
6. The method of embodiment 5, wherein the hydrogel comprises polyacrylamide.
7. The method of embodiment 1, further comprising treating the tissue with a denaturation solution and fixing the tissue before the treating the tissue with the antibody.
8. The method of embodiment 1, wherein the measuring expression levels of the neuronal protein receptor is by signal intensity of antibody-labeled neuronal proteins.
9. The method of any one of embodiments 1-8, wherein the neuronal protein is selected from the group consisting of presynaptic cytomatrix protein Bassoon; glutamate ionotropic N-methyl-D-aspartate (NMDA) receptor type subunit 1 (GluN1); glutamate ionotropic α-amino-3-hydroxy-5-methyl-4-isoxazolepropionic acid (AMPA) type receptor subunit 1 and subunit 2 (GluA1 and GluA2); SH3 and multiple ankyrin repeat domains 3 (SHANK3); α-synuclein (α-Syn); and postsynaptic density protein 95 (PSD-95).
10. The method of any one of embodiments 1-9, wherein the tissue is treated with 2-30 antibodies, wherein each antibody binds to a different epitope.
11. The method of any one of embodiments 1-9, further comprising conducting patch-clamp analysis or analysis of protein expression using artificial intelligence.
12. A kit for analyzing neurons in a brain tissue sample, comprising (a) intracellular solution; (b) cell-staining solution comprising Alexa 488 and biocytin and/or biotin-based dyes; (c) fluorophore-labeled biotin-binding protein; (d) a hydrogel monomer solution containing 30% acrylamide, 10% sodium acrylate, 0.1% bis-acrylamide, and 0.03% VA-044; (e) a denaturation solution containing 6% SDS (w/v), 50 mM sodium sulfite, and 0.02% sodium azide (w/v) in PBS; and (f) antibodies for protein labeling.
13. A system for analyzing neurons in a brain tissue sample, comprising (a) a chamber for housing the brain tissue sample in a physiological solution; (b) one or more micropipettes configured for measuring electrical activity of a set of neurons in the brain tissue sample; (c) a means for visualizing morphology of one or more neurons following injection of a dye; (d) a means for fixing the tissue; (e) a means for expanding the tissue (f) a means for slicing the tissue into sections; and (g) a means for visualizing subcellular location of proteins.
14. The system of embodiment 13, further comprising a computer processor for analyzing and correlating activity of a multiplicity of neurons in the tissue sample; and for correlating neural activity with protein expression of the individual neurons.
15. A method of proteomic imaging of one or more silent synapses, comprising
a) obtaining a whole cell patch clamp recording of a pyramidal neuron; and
b) performing two-photon glutamate uncaging on the neuron to produce an image of the silent synapse.
16. The method of embodiment 15, further comprising performing an Epitope preserving Magnified Analysis of the Proteome (eMAP).
17. The method of embodiment 15 or 16, wherein the one or more silent synapses are located on filopodia.
18. The method of embodiment 17, wherein the filopodia lack AMPAR-mediated synaptic transmission but have NMDAR-mediated synaptic transmission.

### EXAMPLES

### Materials and Methods

### Animals

### Human

Human (Homo sapiens) tissue was acquired through collaboration with the Massachusetts General Hospital (MGH) and the Brigham and Women's Hospital (BWH). For MGH, tissue was obtained as ‘discarded tissue’ from neurosurgical patients in accordance with protocols approved by the Massachusetts General Hospital Internal Review Board (IRB). Patients' consent to surgery and a subset of the resected tissue was considered discarded tissue. Under MGH's IRB-approved protocol, such discarded tissue was available for this specific research project for use without explicit patient consent. For BWH, the protocol was approved by the IRB, and patients provided consent prior to the surgery. At both institutions, non-essential samples were extracted by the supervising neuropathologist per protocol.

Patients were male or female adults aged 23-71 years. Additional patient information is included in FIG. 8. Samples were not allocated to distinct experimental groups and information about the patient was not available until after data acquisition and analysis.

### Mouse

All animal procedures were done in compliance with the NIH and Massachusetts Institute of Technology Committee on Animal Care guidelines. C57BL/6 mice (Charles River Laboratories) were used. Male and female mice were used in approximately equal numbers for all experiments at 8-10 weeks of age. Mice were kept on a 12-hour light/dark cycle and had unrestricted access to food and water.

### Brain Slice Preparation

### Human Slice Preparation

Resected human tissue was considered discarded tissue after being examined by neuropathologists whose main objective was to ensure there was adequate tissue for diagnostic purposes. Neocortical tissue was obtained from the lateral anterior temporal and frontal lobe in patients undergoing resection for medically-intractable epilepsy. The neocortical tissue displayed no known abnormalities at the level of MRI scans, gross inspection, and subsequent microscopic examination as part of the standard neuropathologic assessment of the tissue. Patients undergoing resective surgery were primarily maintained under general anesthesia with propofol and remifentanil or sufentanil. Some cases utilized inhaled anesthetics, such as isoflurane or sevoflurane. For induction of general anesthesia, paralytic agents including rocuronium or succinylcholine as well as fentanyl were typically used. Resection usually occurred within 90 minutes of the start of the procedure.

After resection, tissue was placed in ice-cold cutting solution containing (in mM): sucrose 165, sodium bicarbonate 25, potassium chloride 2.5, sodium phosphate 1.25, calcium chloride 0.5, magnesium chloride 7, glucose 20, HEPES 20, sodium pyruvate 3, and sodium ascorbate 5, 295-305 mOsm, equilibrated with 95% O₂ and 5% CO₂. Samples were transported in sealed conditions for ~20 minutes before being transferred to freshly oxygenated solution. Pia and surface blood vessels that would obstruct slicing were removed. Slicing was performed with a Leica VT1200S vibratome in ice-cold cutting solution. 300 μm-thick slices were incubated for ≥30 minutes at 36° C. in recovery solution containing (in mM): sodium chloride 90, sodium bicarbonate 25, potassium chloride 2.5, sodium phosphate 1.25, calcium chloride 1, magnesium chloride 4, glucose 20, HEPES 20, sodium pyruvate 3, and sodium ascorbate 5, 295-305 mOsm, equilibrated with 95% O₂ and 5% CO₂. Slices were then stored at RT until use. Incubation solutions were replaced every ~8 hours and recordings were performed up to 34 hours after slicing.

### Mouse Slice Preparation

Coronal brain slices (300 μm) containing the primary visual cortex (V1) were prepared from 8- to 10-week-old C57BL/6 mice. Animals were deeply anesthetized with isoflurane prior to decapitation. The brain was removed and sliced with a vibratome (Leica) in ice-cold slicing solution containing (in mM): sucrose 90, NaCl 60, NaHCO₃ 26.5, KCl 2.75, NaH₂PO₄ 1.25, CaCl₂ 1.1, MgCl₂ 5, glucose 9, sodium pyruvate 3, and ascorbic acid 1, saturated with 95% O₂ and 5% CO₂. Slices were incubated in artificial cerebrospinal fluid (aCSF) containing (in mM): NaCl 120, KCl 3, NaHCO₃ 25, NaH₂PO₄ 1.25, CaCl₂ 1.2, MgCl₂ 1.2, glucose 11, sodium pyruvate 3, and sodium ascorbate 1, saturated with 95% O₂ and 5% CO₂ at 35.5° C. for 25-30 min and then stored at RT.

Recording aCSF containing (in mM): sodium chloride 120, potassium chloride 3, sodium bicarbonate 25, sodium phosphate monobasic monohydrate 1.25, calcium chloride 1.2, magnesium chloride 1.2, glucose 11, sodium pyruvate 3, and sodium ascorbate 1, 302-305 mOsm, saturated with 95% O₂ and 5% CO₂.

### Patch-Clamp Recording and Biocytin Filling

Patch-clamp recordings were performed from the soma of pyramidal neurons at 34-36° C. in recording aCSF containing (in mM): sodium chloride 120, potassium chloride 3, sodium bicarbonate 25, sodium phosphate monobasic monohydrate 1.25, calcium chloride 1.2, magnesium chloride 1.2, glucose 11, sodium pyruvate 3 and sodium ascorbate 1, 302-305 mOsm, saturated with 95% O₂ and 5% CO₂.

An Olympus BX-61 microscope with infrared Dodt optics and a 60× water immersion lens (Olympus) was used to visualize cells. Whole-cell current-clamp recordings were performed in bridge mode with a Dagan BVC-700 amplifier with bridge fully balanced. Current and voltage signals were filtered at 10 kHz and digitized at 20 kHz. Patch pipettes were prepared with thin-wall glass (1.5 O.D., 1.1 I.D.). Pipettes had resistances ranging from 3 to 7 MΩ and the capacitance was fully neutralized prior to break in. Series resistances ranged from 7 -17 MΩ. The intracellular solution contained (in mM): potassium gluconate 134, KCl 6, HEPES buffer 10, NaCl 4, Mg₂ATP 4, NaGTP 0.3, phosphocreatine di(tris) 14, 0.1 Alexa 488 (Invitrogen), 5.2 Biocytin (Invitrogen).

During the patch-clamp recording biocytin diffused from the patch pipette to the neuron. A recording time of at least 10 minutes was sufficient to completely fill the axon and the dendrites of large pyramidal neurons in mouse neocortical layers 5 and human neocortical layers 2 and 3. Longer times may be needed for larger neurons or for studies performed at room temperature. Upon completion of the electrophysiological recording, outside-out patch configuration was established by slowly retracing the recording pipette in voltage clamp mode under visual control. The capacitance and input resistance were monitored and loss of capacitive transients with the collapse of the current responses to a straight line ensured resealing of the cell membrane.

The tissue was then processed through a modified protocol for eMAP2, which allowed recovery of biocytin signals for subsequent morphological reconstruction.

### Example 1

The 300 μm slice was transferred in a 15 mL Falcon tube containing 10 mL of 4% PFA in phosphate-buffered saline (PBS) at 4° C. and incubated overnight. Then the slice was incubated in 50 mL Falcon tube containing 40 mL of PBS for at least 24 h at 4° C. The slice was transferred subsequently in a 48-well plate containing 200 μl of washing solution (PBS containing 0.1% (w/v) Triton X-100(PBST) and 0.02% (w/v) sodium azide) on 37° C. shaker for 4 h. PBST was at least once exchanged before adding 20 μl of Streptavidin, Alexa Fluor™ 488 Conjugate (Thermo Fisher, 0.2% (w/v)) and the slice was incubated on 37° C. shaker overnight. The slice was washed 4 times for 10 min each in PBST. This washing process was repeated twice with an interval of 2 hours and the slice was transferred to 4% PFA in phosphate-buffered saline (PBS) at 4° C. and incubated for 8-12h. The slice was switched to PBS at 4° C. for at least 36 h, before performing confocal imaging with excitation at 488 to reveal biocytin filled-neurons.

### Gelation

The slice was incubated in eMAP hydrogel monomer solution (30% acrylamide (A9099, MilliporeSigma, St. Louis, MO, USA), 10% sodium acrylate (408220, MilliporeSigma), 0.1% bis-acrylamide (161-0142, Bio-Rad Laboratories, Hercules, CA, USA), and 0.03% VA-044 (w/v) (Wako Chemicals, Richmond, VA, USA) in PBS) at 4° C. overnight. Then, the slice was carefully placed between two glass slides using Blu-Tack adhesive (Bostik, Essendon Fields, Victoria, Australia) and with the help of ~290 μm thick homemade spacers. The empty space was filled with additional hydrogel monomer solution. The glass slide setup was then placed inside a 50 mL Falcon tube, which was subsequently placed inside Easy-Gel (LifeCanvas Technologies, Cambridge) with nitrogen gas at 37° C. for 3 hours. After gelation, the cartridge was disassembled, and excess gel was trimmed from around the lateral edges of the sample using a razor blade. The resulting tissue-gel hybrid sample was put in PBS with 0.02% sodium azide at 37° C. shaker overnight for hydration.

### Re-Slicing

The tissue gel hybrid was expanded by 1.7×. Confocal imaging is performed to identify the neuron and to decide the side of slicing. A thin layer of Krazy Glue (Elmer's products, Inc. 460 Polaris Parkway Westerville, OH 43082) was applied on a 10 mm non-orienting specimen disc (14048143399, Leica Biosystems, Germany). The originally 300 μm thick slice is placed (neuron closer to the slicing surface) on the glue in the specimen disc and light pressure is applied with a cover slip to make sure uniform application of glue. The tissue gel hybrid is then resliced using a vibratome (VT1200, Leica Biosystems, Germany) in 75 μm (44 μm original) thick slices. From the resulting slices, the ones that contain the neuron of interest are further trimmed with a razor blade to final dimensions of ~3 mm*3 mm*75 μm.

### Denaturation & Immunostaining

The slice was incubated in a denaturation solution [6% SDS (w/v), 0.1 M phosphate buffer, 50 mM sodium sulfite, and 0.02% sodium azide (w/v) in DI water] at 37° C. shaker for 6 h. Subsequently, the samples were transferred in washing solution (PBS containing 0.1% (w/v) Triton X-100 (PBST) and 0.02% (w/v) sodium azide) at 37° C. shaker overnight. The washing solution was exchanged at least once before adding the primary antibodies. The slice was incubated with primary antibodies (typical dilution 1:20) in PBST at 37° C. shaker overnight, followed by washing in PBST at 37° C. for 6 hours with 4 solution exchanges. The same process was repeated for secondary antibodies (typical dilution 1:10).

### Mounting & Image Acquisition

Before each imaging session the slice was incubated in DI water for 1 m to reach final expansion (~3×). Then the expanded specimen was placed between a petri dish and a glass-bottom Willco dish (HBSB-5030; WillCo Wells, Amsterdam, The Netherlands) using glass coverslips as spacers. To prevent the samples from drying up during image acquisition, the void space around the samples was filled up with DI water. Subsequently, the samples were imaged using a Zeiss LSM 900-AS microscope system using a 63× 1.2 water immersion objective or a Leica TCS SP8 microscope system using a 63× 1.2-NA water immersion objective.

### Multiround Staining

The previously stained tissues were incubated in denaturation solution for 6 hours at 37° C., followed by 10 m at preheated 95° C. denaturation solution to remove bound antibodies. The tissue specimen was then washed in PBST at 37° C. overnight. PBST was exchanged at least twice. The samples were imaged on the microscope to confirm the complete loss in the signal from the antibodies. The tissue was then immune-stained as described above.

### Expansion Factor Measurement

To measure the expansion factor, the same cells were imaged at 3 time points: 1. 2-photon imaging of patched cell, 2. gel-tissue hybrid at intermediate expansion, 3. after final expansion (13 human slices). For each cell the mean expansion ratio was calculated from at least 3 measurements among preserved structures along the x, y dimensions (FIG. 4).

### Example 2

### Glutamate Uncaging

A two-photon laser scanning system (Prairie Technologies Ultima) with dual galvanometers and two ultrafast pulsed lasers beams (Mai Tai DeepSee lasers) were used to simultaneously image and uncage glutamate. One path was used to image Alexa 488 at 920 nm. The other path was used to photolyse MNI-caged L-glutamate (Tocris) at 720 nm. Stock MNI solutions (50 mM) were freshly diluted in Mg⁺²-free aCSF to 10 mM and a Picospritzer (General Valve) was used to focally apply the MINI-caged L-glutamate via pressure ejection through a large glass pipette above the slice. Laser beam intensity was independently controlled with electro-optical modulators (model 350-50; Conoptics). Emitted light was collected by GaAsP photomultipliers. Uncaging dwell time was 0.2 ms. A passive 8× pulse splitter in the uncaging path was used to reduce photodamage. Experiments not further analyzed if diffuse signs of photodamage were detected (increase in basal fluorescence, loss of transient signals and/or depolarization).

### Measurement of AMPA-to-NMDA Receptor Ratio at Individual Spines

Uncaging locations were positioned in close vicinity of spines (<0.5 μm) from the tip of individual spine heads in the radial direction. 6-12 spines were individually stimulated at each branch. The uncaging stimulus was delivered in each spine separately (using an isi of 500 ms). Unitary uEPSPs were evoked 20 to 40 times and responses were averaged. Mg²⁺-free aCSF containing 20 μM DNXQ was then washed on for at least 15 minutes. The same uncaging protocol was repeated at the same spines. Care was taken to maintain the initial uncaging locations throughout the experiment.

### Example 3

Measurement of uEPSP Amplitudes

The AMPAR-mediated and NMDAR-mediated uEPSPs amplitude for each spine was measured offline using a custom-written MATLAB code. Briefly, the peak amplitude of each recorded uEPSP was measured during the peak window (50 ms post glutamate uncaging). The width of the window was chosen with consideration of both AMPAR and NMDAR dynamics. Baseline potential was calculated as the average Vm in the 50 ms preceding the evoked uEPSP and was then subtracted from the measurement of the peak uEPSP to provide the individual uEPSP amplitude. Individual uEPSP amplitudes (20-40) were averaged to provide AMPAR-mediated and NMDAR-mediated uEPSPs amplitude for each spine.

### Example 4

### Image Analysis

### Identification of Spines of Interest

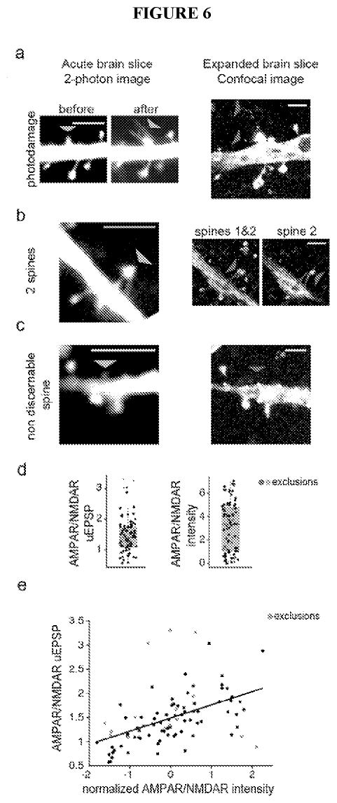

The identification of spines of interest is based on: 1) gross morphology (identification of the branch) and 2) spine imaging (identification of the spines). Both are achieved by comparing the 2-photon (physiology step) and confocal (expansion step) images in terms of geometry, branching points, and relative location to other branches/spines. Examples for identifying the same branches before and after expansion are shown in FIG. 1 and FIG. 2, and for identifying the same spines before and after the expansion are shown in FIGS. 3c, 3d, 3g. The spines, which do not have an unambiguous match in the expanded tissue (FIGS. 6b, 6c) are excluded (see also Correlation analysis).

### Measurement of Signal Intensity in AMPAR and NMDAR Channels

Dendritic spines were analyzed in the biocytin channel using Fiji software. A custom-written macro code was used that first applies a median blur (2 pixels) in the biocytin image and then converts the tip of individual spines to binary masks by thresholding the resulting biocytin image. The rest of the analysis was performed using custom-written MATLAB code. Each image had 4 channels (bassoon, biocytin, NMDAR, AMPAR) and multiple z-slices (z-step of 0.66 μm). The signal intensity in each of the four channels was calculated at every z-slice as the intensity difference of the mask containing the structure of interest and the background. The background intensity was calculated by using the same mask but at random x-y locations of the image to account for the effect of size in the measured intensity among dendritic protrusions. The z-slices that contained the spine of interest were defined as the z-slices where the biocytin intensity signal for a given spine was greater than the median biocytin signal intensity for the same spine from every z-slice+1 SD. The signal intensity for the AMPAR and NMDAR channel for a given spine was finally calculated as the sum of the signal intensity in all the z-slices that contained the respective spine, while the controls (FIGS. 5a and 5b) were calculated by 1000 random combinations of the same number of slices that did not contain the respective spine.

### Z-Score Normalization of Signal Intensity

To pool the data of all cells in a single plot (FIGS. 3j, 6e), the AMPAR:NMDAR signal intensity was z-scored among each subset of cells, which were treated with the same antibodies. The polyclonal antibodies used in these experiments (GluA1 subunit, 182 003 Sysy and GluA2 subunit, 182 103 Sysy) were a complex mixture of several antibodies and commercial vials that differed from each other. Normalization allows to account for differences of the inherent signal intensity of secondary antibodies.

### Correlation Analysis

11 dendritic branches (91 spines, 11 cells, 4 humans), were analyzed using the uncaging experiment and then reconstructed with Patch2MAP the full length of the dendritic segment. From the selection of 2 photon data 4 spines (4/91) was excluded because they showed signs of photodamage (FIG. 6a). From the selection of eMAP data i) 5 spines (5/91) were excluded because the super-resolution morphological imaging revealed that 2 spines instead of 1 were targeted by the 2-photon glutamate uncaging (FIG. 6b). ii) 6 spines (6/91) were excluded because the uncaging location did not correspond to a distinct spine in the expanded branch (FIG. 6c). These exclusions did not change the results presented in the main figures (FIG. 6e).

### Example 5

### Animals

All animal procedures were done in compliance with the NIH and Massachusetts Institute of Technology Committee on Animal care guidelines. The study protocol has been approved by the Massachusetts Institute of Technology Committee on animal care. C57BL/6 mice (Charles River Laboratories) were used for the electrophysiology experiments and viral injections, and Thy1-GFP-M mice (Jackson Laboratory, stock no. 007788) for L5 PN super-resolution experiments. Half of the mice used for the electrophysiology experiments were housed with 2-5 littermates in a large cage (19×10.5×6 in/48×27×15 cm) with a running wheel and plastic shelter tubes. No difference was observed between the conventionally and the enriched housed mice. After virus injection, mice were individually housed for 2 weeks. Male and female mice were used in approximately equal numbers for all experiments at 8-10 weeks of age. Mice were kept on a 12-hour light/dark cycle and had unrestricted access to food and water in a room at 20-22□C and 35-45% humidity. Sample sizes are comparable to or larger than similar studies. No randomization was possible with the study design. Blinding was used in image analysis as detailed in the section below.

### Magnified Analysis of the Proteome

2 male and 2 female Thy1-GFP-M mice were used for the adult L5 proteomic imaging dataset. 1 male and 2 female Thy1-GFP-M mice were used for the P13 proteomic imaging dataset. 2 other Thy1-GFP-M mice at P13 exhibited no GFP-labeled L5 cortical pyramidal neurons in V1. The expression of GFP in the positive P13 mice was very sparse with only 1-2 L5 PNs per hemisphere in V1. This is consistent with the developmental regulation of the expression of GFP in Thy1 transgenic animals.

2 male and 1 female C57/B16 mice injected with a GFP-expressing virus were used for the L2/3 proteomic imaging dataset. These mice were aged 7 weeks at the time of viral injection surgery. All surgeries were performed under aseptic conditions and stereotaxic guidance. Mice were anesthetized with isofluorane (2% induction, 0.75%-1.25% maintenance in 1 l/min oxygen) and secured in a stereotaxic apparatus. Body temperature was maintained with a feedback-controlled heating pad (DC Temperature 21 Control System, FHC). Slow-release buprenorphine (1 mg/kg) was pre-operatively injected subcutaneously. The scalp was cleaned with iodine solution and alcohol. After incision of the scalp, a small burr hole was made using a dental air drill. Mice were injected bilaterally in V1 (stereotactic coordinates: 2.9 mm lateral, 0.4 mm anterior to lambda) with undiluted pAAV1-hSyn-DIO-EGFP virus (addgene Catalog #50457-AAV1) mixed in 1:1 ratio with 1:10000 diluted pENN.AAV.CamKII 0.4.Cre.SV40 virus (addgene Catalog #105558-AAV9). Virus was delivered at a slow rate (max. 50 nL/min) to prevent tissue damage through a small, beveled injection pipette. Virus was injected at an initial depth of 350 μm below the pial surface and moving up 150 μm for a second injection, for a total of approximately 300 nL of injected virus across cortical layer 2/3. The low volume for these experiments was chosen to achieve optimal sparsity for observing pyramidal cell processes. After a five-minute rest, the pipette was slowly withdrawn, and the incision was sutured. Mice were given 2 weeks to recover and for virus expression before perfusion.

Mice were perfused intracardially with cold PBS followed by cold 4% PFA while under deep anesthesia (5% isoflurane). Brains were removed and kept in the same fixative overnight at 4° C., then washed with PBS at 4° C. for 1 day. 1.0 mm coronal slices of primary visual cortex were cut on a vibratome and kept in PBS at 4° C. until the day of processing. Slices were then incubated in the eMAP hydrogel monomer solution: 30% acrylamide (A9099, MilliporeSigma, St. Louis, MO, USA), 10% sodium acrylate (408220, MilliporeSigma), 0.1% bis-acrylamide (161-0142, Bio-Rad Laboratories, Hercules, CA, USA), and 0.03% VA-044 (w/v) (Wako Chemicals, Richmond, VA, USA) in PBS, at 4° C. for 8 to 12 hours. The slices were subsequently mounted between glass slides and sealed in a 50 mL conical tube with nitrogen gas at positive pressure of 10-12 psi for embedding at 35° C. for 3 hours. The excess gel around the slices was then removed. To reach a first expansion stage of 1.7× the slices were then incubated in a solution of 0.02% sodium azide (w/v) in PBS at 37° C. Slices were trimmed to contain only parts of primary visual cortex and further sectioned with a vibratome to 75 μm thickness (corresponding to ~45 μm thickness of the pre-expanded tissue). Slices containing good candidate cells—layer 5 pyramidal neurons whose apical trunk could be reconstructed at its full length in a single slice or at most two consecutive slices—were selected during live low resolution confocal imaging sessions for further processing. These slices were trimmed to smallest possible samples of approximately 1.0 mm in both width and length. Then they were incubated in tissue clearing solution (6% SDS (w/v), 0.1 M phosphate buffer, 50 mM sodium sulfite, 0.02% sodium azide (w/v), pH 7.4) at 37° C. for 6 hours, followed by incubation in preheated clearing solution at 95° C. for 10 min. Cleared samples were thoroughly washed with PBS+0.1% Triton X at 37° C.

Primary antibody staining was performed at 37° C. overnight with the following antibodies: Anti-GFP (Life Technologies A10262), Anti-NMDAR1 (SYSY 114011), Anti-AMPAR1 (SYSY 182003), Anti-Bassoon (SYSY 141004) (typical dilution 1:20). For secondary staining, the following fluorescent antibodies were used: Bassoon—anti-GP-405, GFP—anti-Chicken-488, NMDAR1—anti-Mouse-AF+555, and AMPAR1—anti-Rabbit-AF+647 (typical dilution 1:10). Final expansion was performed just before imaging by putting the trimmed slices in 0.1 mM tris in distilled water. Approximately 4× total linear expansion was achieved and dendritic branches of candidate cells were imaged on a Leica TCS SP8 upright confocal DM6000 microscope equipped with a 63×1.2 NA water immersion objective (300 μm working distance), hybrid detectors, and a white light laser. Leica Application Suite X (LAS X) was used for image acquisition.

123 segments from L5 neurons were imaged. 56 of them originated in basals, 45 in obliques, 20 in trunk and 2 in tuft dendrites. Each image contained a dendritic branch and its dendritic protrusions: spines and filopodia. Dendritic protrusions were analyzed in the GFP channel using ImageJ software. A custom-written macro code was used that first applies a median blur (2 pixels) in the GFP image and then converts the tip of individual dendritic protrusions to binary masks by thresholding the resulting GFP image. The rest of the analysis was performed using custom-written MATLAB code. Each channel (bassoon, GFP, NMDAR, AMPAR) was binarized using intensity thresholds (mean+2 S.D. of the intensity values in each image). Only the protrusions that exhibited a Bassoon-defined presynaptic partner (FIG. 13), qualified as synapses, and were further analyzed. The intensity signal in each of the four channels was finally calculated as the intensity difference of the mask containing the structure of interest and the background. The background intensity was calculated by using the same mask but at random x-y locations of the image to account for the effect of size in the measured intensity among dendritic protrusions. For L2/3 PN experiments, the first 350 background draws from the total 400 (ranked with increasing intensity) were used to calculate the background. This adjustment was used to account for the very high synaptic density in L2/3. Long and thin dendritic protrusion without enlarged head were classified as filopodia (head diameter: neck diameter <1.3 & length: head diameter >3). These definitions were based on previous reports. Dendritic protrusions with an enlarged head were classified as spines. The head and neck diameter were measured perpendicular to long axis of the neck at the widest point. All measurements were made blindly to the bassoon, AMPAR and NMDAR channels.

### Acute Cortical Slice Preparation

Coronal brain slices (300 μm) containing the primary visual cortex (V1) were prepared from 8- to 10-week-old C57BL/6 mice. Animals were deeply anesthetized with isoflurane prior to decapitation. The brain was removed and sliced with a vibratome (Leica) in ice-cold slicing solution containing (in mM): sucrose 90, NaCl 60, NaHCO 3 26.5, KCl 2.75, NaH2PO4 1.25, CaCl2 1.1, MgCl2 5, glucose 9, sodium pyruvate 3, and ascorbic acid 1, saturated with 95% O2 and 5% CO2. Slices were incubated in artificial cerebrospinal fluid (aCSF) containing (in mM): NaCl 120, KCl 3, NaHCO3 25, NaH2PO4 1.25, CaCl2 1.2, MgCl2 1.2, glucose 11, sodium pyruvate 3, and ascorbic acid 1, saturated with 95% O2 and 5% CO2 at 35.5° C. for 25-30 min and then stored at RT. All recordings were performed at 32-35° C. in aCSF. As indicated in the figure legends, Mg²⁺ was omitted from aCSF in some experiments. For focal synaptic stimulation experiments (FIG. 14), the aCSF contained CaCl2 2 and MgCl2 0.

### Patch-Clamp Recording

An Olympus BX-61 microscope with infrared Dodt optics and a 60× water immersion lens (Olympus) was used to visualize cells. Patch-clamp recordings were performed from morphologically and electrophysiologically identified L5b pyramidal cells in V1. Current-clamp recordings were performed in bridge mode with a Dagan BVC-700 amplifier with bridge fully balanced. Current and voltage signals were filtered at 10 kHz and digitized at 20 kHz. Patch pipettes were prepared with thin-wall glass (1.5 O.D., 1.1 I.D.). Pipettes had resistances ranging from 3 to 7 MΩ and the capacitance was fully neutralized prior to break in. Series resistances ranged from 6-25 MΩ. The intracellular solution contained (in mM): potassium gluconate 134, KCl 6, HEPES buffer 10, NaCl 4, Mg2ATP 4, NaGTP 3, phosphocreatine di (tris) 14. Depending on the experiment, 0.1 Alexa 488 (Invitrogen) or 0.05 Alexa 594 (Invitrogen) and 0.2 Fluo-4 mM (Invitrogen) were added to the intracellular solution. Bruker Prairie View Software was used for the data acquisition.

### Two-Photon Imaging and Uncaging

A two-photon laser scanning system (Prairie Technologies Ultima) with dual galvanometers and two ultrafast pulsed lasers beams (Mai Tai DeepSee lasers) were used to simultaneously image and uncage glutamate. One path was used to image Alexa 488 at 920 nm. The other path was used to photolyse MNI-caged L-glutamate (Tocris) at 720 nm. Stock MNI solutions (50 mM) were freshly diluted in aCSF to 10 mM and a Picospritzer (General Valve) was used to focally apply the MNI-caged L-glutamate via pressure ejection through a large glass pipette above the slice. Laser beam intensity was independently controlled with electro-optical modulators (model 350-50; Conoptics). Emitted light was collected by GaAsP photomultipliers. Uncaging dwell time was 0.2 ms. A passive 8X pulse splitter in the uncaging path was used to reduce photodamage. Experiments were terminated if signs of photodamage were detected (increase in basal fluorescence, loss of transient signals and/or depolarization). Filopodia were identified by their long and thin morphology without an enlarged head (head: neck diameter<1.3 & length: head diameter>3). All uncaging experiments on filopodia were conducted with a neighboring control spine. The laser power of the uncaging laser was adjusted to elicit somatic responses when neighboring spines were targeted. Uncaging locations were manually positioned in close vicinity (<0.5 μm) from the tip of the spine or filopodia. The uncaging locations were manually readjusted if necessary between individual trials. Care was taken to ensure that the selected spines or filopodia were well isolated (no spines within 1 μm laterally and no spines above or below in z). To isolate NMDA-mediated EPSPs in FIG. 14, experiments were conducted as described for glutamate uncaging above, except stock MNI-caged glutamate solution (50 mM) was freshly diluted in Mg²⁺-free aCSF to 10 mM and Mg+2-free aCSF containing 20 μM DNXQ was washed on to the slice for at least 15 minutes.

### Focal Synaptic Stimulation

Patch-clamp recordings were acquired in control aCSF. After whole cell configuration, modified aCSF containing (in mM): NaCl 120, KCl 3, NaHCO3 25, NaH2PO4 1.25, CaCl2 2, MgCl2 0, glucose 11, sodium pyruvate 3, ascorbic acid 1 and, DNQX 0.02 was washed on the slice for at least 15 min. Theta-glass bipolar stimulating electrodes filled with ACSF containing 0.05 Alexa 594 for visualization were positioned near dendritic filopodia (~10 μm) under two-photon guidance. Stimuli were delivered with an AMPI Isoflex isolator. Stimulus intensity was increased until an action potential was initiated and then intensity was decreased to generate EPSPs below AP threshold. Relative changes in fluorescence (F/F) of the Ca²⁺ indicator Fluo-4 were measured simultaneously at the tip of the filopodium and the parent branch. Trials in which synaptic stimulation resulted in somatic APs were excluded from further analysis. Only the trials with no detectable change in fluorescence of the parent branch were further analyzed.

### Plasticity Studies

For plasticity induction, two-photon glutamate uncaging at filopodia or spines of interest was followed in time (10 ms) by a backpropagating action potential, which was generated by injecting a 2 ms current pulse of 1.2-2 nA at the soma. This pairing was repeated 40 times at 2 Hz. Two control protocols were used at filopodia: 1) pre alone: two-photon glutamate uncaging at filopodia was repeated 40 times at 2 Hz with no somatic action potentials; and 2) post alone: without any caged glutamate present, two-photon laser pulses at filopodia were followed (10 ms) by a backpropagating action potential, repeated 40 times at 2 Hz. A longer STDP pairing protocol consisting of 90 repeats at 2 Hz was also used at spines (FIG. 22).

For plasticity experiments in filopodia, a neighboring spine was first stimulated separately with control pulses and the uncaging laser power was adjusted to yield large (0.2-1.2 mV) somatic EPSPs. Plasticity induction was applied using the same laser power. This was followed by test stimulations of filopodia using the same laser power ~3-5 min after the induction. Filopodia were excluded if they moved close (<0.1 μm) to other neighboring spines due to shape or size changes throughout the course of the experiment. The magnitude of plasticity was quantified as the average change in EPSP amplitude after the plasticity protocol. The percentage change was not calculated to avoid division by small numbers (initial EPSP=~0 mV). The change in the protrusion length was used as a structural metric of the plasticity experiment because the estimation of spine volume is complicated by the increase of Alexa Fluor 488 fluorescence during the course of the experiment. Morphological and distance measurements were performed using ImageJ/FIJI (National Institutes of Health) on two-dimensional images collected during the experiment.

For plasticity experiments in spines, a second spine on a different branch was used as a control and was not stimulated during plasticity induction. Both test and control spines were stimulated with test pulses before and after the plasticity protocol induction. Control filopodia were tested in separate cells from test filopodia. Spines and control filopodia were followed up to 20-30 min after the plasticity protocol induction. Care was taken not to move the uncaging location closer to the spine head during the experiment to avoid artificial increases in EPSP amplitudes. For the plasticity induction protocol with 90 repetitions, both test and control spines showed a slight decrease in EPSP amplitude, consistent with previous reports. The magnitude of plasticity was quantified as the average change in EPSP amplitude after the plasticity protocol.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of analyzing neurons in a subject with normal neurologic function or a subject having neurological and/or neuropsychiatric disease, comprising
a. obtaining a brain tissue sample from the subject, wherein the brain tissue sample comprises viable neuronal tissue;
b. patch-clamping one or more neurons in the viable neuronal tissue to record their individual electrical activity, wherein the one or more neurons are alive during patch-clamping;
c. filling the patch-clamped neurons with a dye to identify morphological features of the one or more neurons in the neuronal tissue; and
d. processing the brain tissue sample to physically expand it; and
e. treating the brain tissue sample with an antibody that enables detection of a protein in the one or more neurons.

2. The method of claim 1, wherein the neurological and/or neuropsychiatric disease is selected from attention-deficit/hyperactivity disorder (ADHD), autism spectrum disorder (ASD), communication disorders, intellectual developmental disorder, intellectual disability, learning disorder, disruptive mood dysregulation disorder, dementias (Alzheimer's disease, vascular dementia, Lewy body dementia, frontotemporal lobar degeneration), brain cancer, glioblastoma, epilepsy, movement disorders (Parkinson's disease, Huntington's disease, multiple system atrophy, progressive supranuclear palsy, restless leg syndrome, tremor, Tourette syndrome), stroke, Transient Ischemic Attack, functional neurological disorders, anxiety disorders, mood disorders, psychotic disorders, eating disorders, impulse control and addiction disorders, and personality disorders.

3. The method of claim 1, wherein the neurological and/or neuropsychiatric disease is glioblastoma or Alzheimer's disease.

4. The method of claim 1, wherein the dye comprises biotin or biotin-based dyes and biotin binding proteins (e.g. fluorophore-labeled streptavidin).

5. The method of claim 1, further comprising treating the tissue with formaldehyde and/or a hydrogel.

6. The method of claim 5, wherein the hydrogel comprises polyacrylamide.

7. The method of claim 1, further comprising treating the tissue with a denaturation solution and fixing the tissue before the treating the tissue with the antibody.

8. The method of claim 1, wherein the measuring expression levels of the neuronal protein receptor is by signal intensity of antibody-labeled neuronal proteins.

9. The method of any one of claims 1-8, wherein the neuronal protein is selected from the group consisting of presynaptic cytomatrix protein Bassoon; glutamate ionotropic N-methyl-D-aspartate (NMDA) receptor type subunit 1 (GluN1); glutamate ionotropic α-amino-3-hydroxy-5-methyl-4-isoxazolepropionic acid (AMPA) type receptor subunit 1 and subunit 2 (GluA1 and GluA2); SH3 and multiple ankyrin repeat domains 3 (SHANK3); α-synuclein (α-Syn); and postsynaptic density protein 95 (PSD-95).

10. The method of any one of claims 1-9, wherein the tissue is treated with 2-30 antibodies, wherein each antibody binds to a different epitope.

11. The method of any one of claims 1-9, further comprising conducting patch-clamp analysis or analysis of protein expression using artificial intelligence.

12. A kit for analyzing neurons in a brain tissue sample, comprising (a) intracellular solution; (b) cell-staining solution comprising Alexa 488 and biocytin and/or biotin-based dyes; (c) fluorophore-labeled biotin-binding protein; (d) a hydrogel monomer solution containing 30% acrylamide, 10% sodium acrylate, 0.1% bis-acrylamide, and 0.03% VA-044;
(e) a denaturation solution containing 6% SDS (w/v), 50 mM sodium sulfite, and 0.02% sodium azide (w/v) in PBS; and (f) antibodies for protein labeling.

13. A system for analyzing neurons in a brain tissue sample, comprising (a) a chamber for housing the brain tissue sample in a physiological solution; (b) one or more micropipettes configured for measuring electrical activity of a set of neurons in the brain tissue sample; (c) a means for visualizing morphology of one or more neurons following injection of a dye; (d) a means for fixing the tissue; (e) a means for expanding the tissue (f) a means for slicing the tissue into sections; and (g) a means for visualizing subcellular location of proteins.

14. The system of claim 13, further comprising a computer processor for analyzing and correlating activity of a multiplicity of neurons in the tissue sample; and for correlating neural activity with protein expression of the individual neurons.

15. A method of proteomic imaging of one or more silent synapses, comprising
a) obtaining a whole cell patch clamp recording of a pyramidal neuron; and
b) performing two-photon glutamate uncaging on the neuron to produce an image of the silent synapse.

16. The method of claim 15, further comprising performing an Epitope preserving Magnified Analysis of the Proteome (eMAP).

17. The method of claim 15 or 16, wherein the one or more silent synapses are located on filopodia.

18. The method of claim 17, wherein the filopodia lack AMPAR-mediated synaptic transmission but have NMDAR-mediated synaptic transmission.
